SEQUENCE LISTING (61 sequences)

SEQ ID NO: 1 - DNA, 2202 bp, Artificial
  Cap2/5 - g236
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggtggtgccggtgccggtgccggtggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 2 - protein, 733 aa, Artificial
  Synthetic Construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGGAGAGAGGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 3 - DNA, 2202 bp, Artificial
  Cap2/5 - g267
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccggtggtgccggtgccggtgccggtgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 4 - protein, 733 aa, Artificial
  Synthetic Construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSGGAGAGAGVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 5 - DNA, 2202 bp, Artificial
  Cap2/5 - g382
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacaggtggtgccggtgccggtgccggtgaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 6 - protein, 733 aa, Artificial
  Synthetic Construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTGGAGAGAGENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 7 - DNA, 2202 bp, Artificial
  Cap2/5 - 454
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacggtggtgccggtgccggtgccggtactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 8 - protein, 733 aa, Artificial
  Synthetic Construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGGAGAGAGTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 9 - DNA, 2202 bp, Artificial
  Cap2/5 - 467
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacggtggtgccggtgccggtgccggtctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 10 - protein, 733 aa, Artificial
  Synthetic Construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNGGAGAGAGLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 11 - DNA, 2202 bp, Artificial
  Cap2/5 - 502
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcggtggtgccggtgccggtgccggtgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 12 - protein, 733 aa, Artificial
  Synthetic Construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSGGAGAGAGAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 13 - DNA, 2202 bp, Artificial
  Cap2/5 - 663
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcgggtggtgccggtgccggtgccggtgacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 14 - protein, 733 aa, Artificial
  Synthetic Construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSGGAGAGAGDVPVSSFITQYSTGQVTVEMEWELKKENSKRWNPEIQYTNNYNDPQFVDFAPDSTGEYRTTRPIGTRYLTRPL

SEQ ID NO: 15 - DNA, 2202 bp, Artificial
  Cap2/5 - 708
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgaccccggtggtgccggtgccggtgccggtcagtttgtggactttgccccggacagcaccggggaatacagaaccaccagacctatcggaacccgataccttacccgacccctttaa

SEQ ID NO: 16 - protein, 733 aa, Artificial
  Synthetic Construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPGGAGAGAGQFVDFAPDSTGEYRTTRPIGTRYLTRPL

SEQ ID NO: 17 - DNA, 2202 bp, Artificial
  Cap2/5 - 751
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgaccccttggtggtgccggtgccggtgccggttaa

SEQ ID NO: 18 - protein, 733 aa, Artificial
  Synthetic Construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPLGGAGAGAG

SEQ ID NO: 19 - protein, 733 aa, Artificial; identical to SEQ ID NO: 2
  Cap2/5 - 236

SEQ ID NO: 20 - protein, 733 aa, Artificial; identical to SEQ ID NO: 4
  Cap2/5 - 267

SEQ ID NO: 21 - protein, 733 aa, Artificial; identical to SEQ ID NO: 6
  Cap2/5 - 382

SEQ ID NO: 22 - protein, 733 aa, Artificial; identical to SEQ ID NO: 8
  Cap2/5 - 454

SEQ ID NO: 23 - protein, 733 aa, Artificial; identical to SEQ ID NO: 10
  Cap2/5 - 467

SEQ ID NO: 24 - protein, 733 aa, Artificial; identical to SEQ ID NO: 12
  Cap2/5 - 502

SEQ ID NO: 25 - protein, 733 aa, Artificial; identical to SEQ ID NO: 14
  Cap2/5 - 663

SEQ ID NO: 26 - protein, 733 aa, Artificial; identical to SEQ ID NO: 16
  Cap2/5 - 708

SEQ ID NO: 27 - protein, 733 aa, Artificial; identical to SEQ ID NO: 18
  Cap2/5 - 751

SEQ ID NO: 28 - protein, 8 aa, Artificial
  immune evasion repeat sequence
GGAGAGAG

SEQ ID NO: 29 - DNA, 54 bp, Artificial
  Pr243
gattccacgtggatgggtggtgccggtgccggtgccggtggggacagagtcgtc

SEQ ID NO: 30 - DNA, 54 bp, Artificial
  Pr244
gacgactctgtccccaccggcaccggcaccggcaccacccatccacgtggaatc

SEQ ID NO: 31 - DNA, 52 bp, Artificial
  Pr245
caaaagcggctccggtggtgccggtgccggtgccggtgtcgacggaagcaac

SEQ ID NO: 32 - DNA, 52 bp, Artificial
  Pr246
gttgcttccgtcgacaccggcaccggcaccggcaccaccggagccgcttttg

SEQ ID NO: 33 - DNA, 55 bp, Artificial
  Pr247
gaaccgcgacaacacaggtggtgccggtgccggtgccggtgaaaatcccaccgag

SEQ ID NO: 34 - DNA, 55 bp, Artificial
  Pr248
ctcggtgggattttcaccggcaccggcaccggcaccacctgtgttgtcgcggttc

SEQ ID NO: 35 - DNA, 55 bp, Artificial
  Pr249
ggcaacaactttgagggtggtgccggtgccggtgccggttttacctacaactttg

SEQ ID NO: 36 - DNA, 55 bp, Artificial
  Pr250
caaagttgtaggtaaaaccggcaccggcaccggcaccaccctcaaagttgttgcc

SEQ ID NO: 37 - DNA, 54 bp, Artificial
  Pr251
gtgagcacaaataacggtggtgccggtgccggtgccggtactggcggagtccag

SEQ ID NO: 38 - DNA, 54 bp, Artificial
  Pr252
ctggactccgccagtaccggcaccggcaccggcaccaccgttatttgtgctcac

SEQ ID NO: 39 - DNA, 54 bp, Artificial
  Pr253
cagttcaacaagaacggtggtgccggtgccggtgccggtctggccgggagatac

SEQ ID NO: 40 - DNA, 54 bp, Artificial
  Pr254
gtatctcccggccagaccggcaccggcaccggcaccaccgttcttgttgaactg

SEQ ID NO: 41 - DNA, 54 bp, Artificial
  Pr255
cgcgccagtgtcagcggtggtgccggtgccggtgccggtgccttcgccacgacc

SEQ ID NO: 42 - DNA, 54 bp, Artificial
  Pr256
ggtcgtggcgaaggcaccggcaccggcaccggcaccaccgctgacactggcgcg

SEQ ID NO: 43 - DNA, 55 bp, Artificial
  Pr257
ctatgatcttcaacagcggtggtgccggtgccggtgccggtcagccggcgaaccc

SEQ ID NO: 44 - DNA, 55 bp, Artificial
  Pr258
gggttcgccggctgaccggcaccggcaccggcaccaccgctgttgaagatcatag

SEQ ID NO: 45 - DNA, 54 bp, Artificial
  Pr259
ggcaccaccgccacgggtggtgccggtgccggtgccggttacctcgagggcaac

SEQ ID NO: 46 - DNA, 54 bp, Artificial
  Pr260
gttgccctcgaggtaaccggcaccggcaccggcaccacccgtggcggtggtgcc

SEQ ID NO: 47 - DNA, 53 bp, Artificial
  Pr261
ccggcacgtacaacggtggtgccggtgccggtgccggtctccaggaaatcgtg

SEQ ID NO: 48 - DNA, 53 bp, Artificial
  Pr262
cacgatttcctggagaccggcaccggcaccggcaccaccgttgtacgtgccgg

SEQ ID NO: 49 - DNA, 52 bp, Artificial
  Pr263
caccagcttctcgggtggtgccggtgccggtgccggtgacgtgcccgtcagc

SEQ ID NO: 50 - DNA, 52 bp, Artificial
  Pr264
gctgacgggcacgtcaccggcaccggcaccggcaccacccgagaagctggtg

SEQ ID NO: 51 - DNA, 56 bp, Artificial
  Pr265
caactacaacgaccccggtggtgccggtgccggtgccggtcagtttgtggactttg

SEQ ID NO: 52 - DNA, 56 bp, Artificial
  Pr266
caaagtccacaaactgaccggcaccggcaccggcaccaccggggtcgttgtagttg

SEQ ID NO: 53 - DNA, 57 bp, Artificial
  Pr280
cttacccgaccccttggtggtgccggtgccggtgccggttaagacccagctttcttg

SEQ ID NO: 54 - DNA, 57 bp, Artificial
  Pr281
caagaaagctgggtcttaaccggcaccggcaccggcaccaccaaggggtcgggtaag

SEQ ID NO: 55 - DNA, 21 bp, Artificial
  Pr286
ggactccaagccttccacctc

SEQ ID NO: 56 - DNA, 20 bp, Artificial
  Pr287
gccaacaacctcacctccac

SEQ ID NO: 57 - DNA, 20 bp, Artificial
  Pr288
ccagggcagcaacacctatg

SEQ ID NO: 58 - DNA, 19 bp, Artificial
  Pr299
gcaggtcaccgtggagatg

SEQ ID NO: 59 - DNA, 22 bp, Artificial
  Pr315
gctgctttaatgcctttgtatc

SEQ ID NO: 60 - DNA, 20 bp, Artificial
  Pr316
aatgaaagccatacgggaag

SEQ ID NO: 61 - protein, 725 aa, Artificial
  AAV2/5
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

(19) United States
(12) Patent Application Publication
(10) Pub. No.: US 2011/0171262 A1
(43) Pub. Date: Jul. 14, 2011

(54) PARVOVIRAL CAPSID WITH INCORPORATED GLY-ALA REPEAT REGION

(76) Inventors: Andrew Christian Bakker, Utrecht (NL); Valerie Sier-Ferreira, Hoofddorp (NL); Sebastiaan Menno Bosma, Heerhugowaard (NL)

(21) Appl. No.: 12/999,860

(22) PCT Filed: Jun. 17, 2009
(86) PCT No.: PCT/NL2009/050352

§ 371 Date: Mar. 21, 2011

Related U.S. Application Data
(60) Provisional application No. 61/073,295, filed on Jun. 17, 2008.

(30) Foreign Application Priority Data
Jun. 17, 2008 (EP) 08158418.7

(51) Int. Cl. A61K 39/23; C12N 15/63; C12N 5/10; C12N 15/85; C12N 7/00; A61K 35/76; A61P 37/06

(52) U.S. Cl. 424/233.1; 435/320.1; 435/325; 435/348; 435/235.1; 424/93.6

(57) ABSTRACT

Parvoviral capsid with incorporated Gly-Ala repeat region The present invention provides a nucleic acid construct comprising a nucleic acid sequence encoding a parvoviral VP1, VP2 and VP3 capsid proteins comprising an immuno evasion repeat sequence. In addition, the present invention provides a cell comprising such construct, a parvoviral virion comprising a capsid protein that comprises an immune evasion repeat sequence, use of that parvoviral virion in gene therapy and a pharmaceutical composition comprising such parvoviral virion.

### FIELD OF THE INVENTION

The present invention relates to the production of parvovirus vectors, especially to the production of recombinant adeno-associated viruses (rAAV), the capsid proteins of which do not trigger an adaptive immune response when inserted into a cell of a patient. In addition this invention relates to cap proteins comprising a Gly-Ala repeat region and to nucleic acid constructs encoding therefor.

### BACKGROUND OF THE INVENTION

One of the obstacles to overcome in gene therapy is the T-cell mediated destruction of cells that are infected by adeno-associated virus (AAV). Following infection of a cell, the AAV capsid is processed by the proteasome and small AAV capsid specific peptides are presented on the cell surface by MHC complexes. Cytotoxic T-cells specific for these peptides can then recognize the cells and kill them. Also, loss of transgene expression after delivery by AAV-based vectors may be mediated by an antibody response.

The Glycine-Alanine repeat (GAr) region of the Epstein-Barr virus nuclear antigen-1 (EBNA1) is a repeat of 60 to 300 amino acids long, depending on the Epstein-Barr virus (EBV) strain. Inhibition of proteasomal degradation of linked antigens by the GAr region was originally disclosed by Masucci and co-workers (Levitskaya et al. (1995) Nature: 685-688; Levitskaya et al. (1997) PNAS USA:12616-12621). In EBV, EBNA1 prevents protein degradation by the proteasome system and thereby prevents presentation of peptide fragments on major histocompatibility complex class 1 (MHC1) or human leukocyte antigen class 1 (HLA1) and a subsequent T-cell response. Although the exact functionality of GAr is yet to be determined, it is hypothesized that GAr prevents degradation of proteins, because apolar amino acids (glycine and alanine) of the domain cause the proteasome to slip over the repeat region. This could prevent proper breakdown of the protein by the proteasome and thus presentation of AAV peptides by MHC complexes. It was shown that introduction of this long repeat region in other proteins also resulted in reduced protein breakdown.

In WO 97/46573 it is disclosed that a minimum of about 30 amino acids of a Gly-Ala repeat domain in a foreign protein is capable of inhibiting the cytopathic T lymphocyte immune response to the foreign protein, e.g. in gene therapy. A Gly-Ala repeat sequence of less than 35 amino acids is considered too small to sufficiently inhibit toxicity.

U.S. Pat. No. 5,833,991 discloses glycine-rich repeat sequences that upon insertion into a protein which is normally antigenic confers upon the recombinant protein the ability to evade the immune system. U.S. Pat. No. 5,833,991 suggests to use the glycine-rich repeat sequence for viral vector-mediated gene transfer, thereby aiming to avoid an undesired immune response directed to antigenic structural proteins of transfer vectors.

Zaldumbide and Hoeben (Gene Therapy 2008:239-246) reviewed some of the options to blunt acquired immune responses to transgene-encoded polypeptides in gene therapy. One of the options they suggested is the generation of proteins that are protected from proteasomal degradation by fusion of a GAr region or glycine, glutamine and glutamic acid residues (GZr) repeats to the protein that is to be protected. However, at the present the insertion of a long GAr region in an capsid protein is not feasible. The authors state that it remains to be established whether a GAr as short as 24 amino acids is sufficient for immune evasion. Furthermore, the authors disclose that degradation of normal proteins by the proteasome is only responsible for a small fraction of the antigenic peptides expressed on the cell surface and most of the antigenic peptides are derived from defective products of protein synthesis.

Therefore, there is a need for gene therapy vectors, in particular parvoviral vectors, that result in reduced immune responses.

### DESCRIPTION OF THE INVENTION

### Definitions

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

“Expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons. The term “expression control sequence” is intended to include, at a minimum, a sequence whose presence are designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which effect the translation, e.g., Kozak sequences, are known in insect cells. Expression control sequences can be of such nature as to modulate the nucleotide sequence to which it is operably linked such that lower expression levels or higher expression levels are achieved.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells.

The terms “substantially identical”, “substantial identity”, or “essentially similar” or “essential similarity” means that two peptide or two nucleotide sequences, when optimally aligned, such as by the programs GAP or BESTFIT using default parameters, share at least a certain percentage of sequence identity as defined elsewhere herein. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). It is clear than when RNA sequences are said to be essentially similar or have a certain degree of sequence identity with DNA sequences, thymine (T) in the DNA sequence is considered equal to uracil (U) in the RNA sequence. Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA or the open-source software Emboss for Windows (current version 2.7.1-07). Alternatively percent similarity or identity may be determined by searching against databases such as FASTA, BLAST, etc.

The terms “transduction”, “transfection”, “transformation” and “infection” are herein used interchangeably and are intended to mean introduction into a cell of nucleic acid material using a viral vector, a (parvoviral) virion or any other means of transfer.

### DETAILED DESCRIPTION OF THE INVENTION

The present invention relates to the use animal parvoviruses, in particular dependoviruses such as infectious human or simian AAV, and the components thereof (e.g., an animal parvovirus genome) for use as vectors for introduction and/or expression of nucleic acids in mammalian cells. In particular, the invention relates to a parvoviral virion that shows transduction efficiency in vivo and evades of the cytotoxic T lymphocytes against the capsid protein.

Viruses of the Parvoviridae family are small DNA animal viruses. The family Parvoviridae may be divided between two subfamilies: the Parvovirinae, which infect vertebrates, and the Densovirinae, which infect insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 2, 3A, 3B, 5, and 6) or primates (e.g., serotypes 1 and 4, which are thought to have been originated from monkeys, but also infect humans), and related viruses that infect other warm-blooded animals (e.g., bovine, canine, equine, and ovine adeno-associated viruses). Further information on AAV serotypes and on strategies for engineering hybrid AAV vectors derived from AAV serotypes is described in Wu et al. (2006, Molecular Therapy 14:316-327). For convenience the present invention is further exemplified and described herein by reference to AAV. It is however understood that the invention is not limited to AAV but may equally be applied to hybrid AAV vectors derived from two or more different AAV serotypes and to other parvoviruses and hybrids thereof.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins, Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

According to the invention, there is thus provided a nucleic acid construct comprising a nucleotide sequence encoding parvoviral VP1, VP2, and VP3 capsid proteins, wherein the nucleotide sequence comprises at least one in frame insertion of a sequence coding for an immune evasion repeat. Preferably, the immune evasion repeat is an amino acid sequence that comprises 1, 2 or 3 units of a formula (Glyₘ-Xaa₁-Glyₙ-Xaa₂-Glyₚ-Xaa₃-Gly_{q}) wherein m and q are each independently 0, 1 or 2, wherein n, and p are each independently 1, 2 or 3, wherein m, q, n and p are chosen such that the immune evasion repeat consists of at least 8 amino acids, and wherein each of Xaa₁, Xaa₂, and Xaa₃ are independently of each other Ala or Val or another small hydrophobic amino acid residue, for example such as Ile, Leu, Met, Phe or Pro. More preferably Xaa is a small hydrophobic amino acid residue selected from the group consisting of Ala, Val, Ile and Leu. Most preferably Xaa is a small hydrophobic amino acid residue selected from the group consisting of Ala and Val. Of the small hydrophobic amino acids, the smaller ones are more preferred for use in the invention than the larger ones. It is understood that where the immune evasion repeat comprises more that one unit of the formula, the amino acid sequences of the individual units may differ from each other or they may be identical. Two units may be identical and a third unit different from those two.

The immune evasion repeat may be 8 amino acids in length. The repeat may be nine, ten, eleven, twelve amino acids or longer in length.

In one embodiment of the invention the immune evasion repeat comprises one unit of the formula, wherein one of m, n or p is 2 and the other two of m, n or p are 1, and q is 1, and each of Xaa₁, Xaa₂, and Xaa₃ is Ala. In a preferred embodiment m=2, n=1, p=1, and q=1.

Sharipo et al. (2001 FEBS Letters 499:137-142) have investigated the capacity of various octamer immune evasion repeat sequences (also known as Gly-Ala repeat or GAr) to act as cis-inhibitor of ubiquitin-proteasome dependent proteolysis. They suggest a model where inhibition requires the interaction of at least three alanine residues of the GAr in a beta-strand conformation with adjacent hydrophobic binding pockets of a putative receptor. Preferably, immune evasion repeat sequences comprise at least 8 amino acids. Preferred immune evasion repeat sequences of the invention are: Gly-Gly-Xaa₁-Gly-Xaa₂-Gly-Xaa₃-Gly; Gly-Xaa₁-Gly-Xaa₂-Gly-Gly-Xaa₃-Gly; Gly-Xaa₁-Gly-Gly-Xaa₂-Gly-Xaa₃-Gly; Xaa₁-Gly-Gly-Xaa₂-Gly-Gly-Xaa₃-Gly; Gly-Xaa₁-Gly-Xaa₂-Gly-Gly-Gly-Xaa₁ and Gly-Xaa₁-Gly-Gly-Gly-Xaa₂-Gly-Xaa₃, wherein all Xaa are independently of each other alanine or valine or another small hydrophobic amino acid such as Ile, Leu, Met, Phe or Pro. Preferably, the immune evasion repeat sequence is any one of the group consisting of Gly-Gly-Ala-Gly-Ala-Gly-Ala-Gly; Gly-Gly-Val-Gly-Val-Gly-Val-Gly; Gly-Gly-Ala-Gly-Ala-Gly-Ala-Gly-Gly-Gly-Ala-Gly-Ala-Gly-Ala-Gly-Gly-Gly-Ala-Gly-Ala-Gly-Ala-Gly.

The at least one sequence coding for an immune evasion repeat may be present in the part of the nucleotide sequence coding for the VP1, VP2 or VP3 capsid protein. Preferably, the at least one sequence coding for an immune evasion repeat is present in the part of the nucleotide sequence coding for the VP3 capsid protein. The at least one sequence coding for an immune evasion repeat may be incorporated into a VP1, VP2 or VP3 capsid protein at any position. Preferably, however, the insertion of the immune evasion repeat does not interfere with at least one of efficiency of virion production and efficient transduction of target cells (i.e. infectivity).

The insertion may cause immune evasion in the sense that it leads to the reduction or absence of an adaptive immune response (that may take place when the immune evasion repeat is not present). The insertion may cause evasion because of a reduction, or more preferably absence, of presentation of processed capsid proteins by the virion infected cell, thereby preventing cytotoxic T lymphocytes from recognising and killing a cell infected by a parvoviral vector of the invention. The insertion may cause evasion in the sense that it leads to a reduced or no antibody response, for example the reduction or absence of neutralizing antibodies. Preferably also, the insertion causes evasion or at least a reduction in cytotoxic T lymphocyte response(s) against the virion infected target cells. The insertion may lead to neutralizing antibodies being raised which do not prevent (or reduce the extent of inhibition of) subsequent infection of cells by a gene therapy vector such that a gene therapy vector may be used for readministration.

In one embodiment of the invention, a sequence coding for an immune evasion repeat as defined above is present in at least one position in the VP3 capsid protein that is immediately N-terminal to an amino acid in the VP3 capsid protein that corresponds to an amino acid position selected from the group consisting of amino acid positions 226, 255, 377, 444, 453, 488, 652, 697 and 726 of the AAV2/5 hybrid capsid protein (SEQ ID NO: 61). More preferably, the immune evasion repeat is present in at least one position in the VP3 capsid protein that is immediately N-terminal to an amino acid in the VP3 capsid protein that corresponds to an amino acid position selected from the group consisting of amino acid positions 255, 377, 444, 652, 697 and 726 of the AAV2/5 hybrid capsid protein. Most preferably, the immune evasion repeat is present in at least one position in the VP3 capsid protein that is immediately N-terminal to an amino acid in the VP3 capsid protein that corresponds to an amino acid position selected from the group consisting of amino acid positions 255, 377 and 444 of the AAV2/5 hybrid capsid protein, of which amino acid positions 255 is most preferred. Since the genomic organisation of all known AAV serotypes is very similar, the skilled man in the art knows how to convert these positions to suitable positions in other serotypes or other parvoviral vectors, see e.g. FIG. 1. In another embodiment, a sequence coding for an immune evasion repeat is present at the carboxy terminal side of VP3.

Parvoviral sequences that may be used in the present invention can be derived from the genome of any AAV serotype. Generally, the AAV serotypes have genomic sequences of significant identity and/or similarity at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chiorini et al. (1997, J. Vir. 71: 6823-33); Srivastava et al. (1983, J. Vir. 45:555-64); Chiorini et al. (1999, J. Vir. 73:1309-1319); Rutledge et al. (1998, J. Vir. 72:309-319); and Wu et al. (2000, J. Vir. 74: 8635-47). Human or simian adeno-associated virus (AAV) serotypes are preferred sources of AAV nucleotide sequences for use in the context of the present invention, more preferably AAV serotypes which normally infects humans (e.g., serotypes 1, 2, 3A, 3B, 4, 5, and 6) or primates (e.g., serotypes 1 and 4). AAV serotypes 2 and 5 are particularly preferred.

In a particular preferred embodiment, the nucleic acid sequence of the invention has at least 50%, 55%, 60%, 65%, 70%, 75%, 80%, 85%, 90%, 95%, 97%, 99%, 100% sequence identity with any one of SEQ ID NO: 1, 3, 5, 7, 9, 11, 13, 15 or 17 encoding for an amino acid sequence that has at least 60%, 65%, 70%, 75%, 80%, 85%, 90%, 95%, 97%, 99%, 100% sequence identity with any one of SEQ ID NO: 2, 4, 6, 8, 10, 12, 14, 16 or 18.

In one embodiment, the nucleotide sequence of the invention is operably linked to expression control sequences for expression in a mammalian or insect cell.

Preferably the nucleotide sequence of the invention encoding parvoviral VP1, VP2, and VP3 capsid proteins is operably linked to expression control sequences for expression in an insect cell. These expression control sequences will at least include a promoter that is active in insect cells. Techniques known to one skilled in the art for expressing foreign genes in insect host cells can be used to practice the invention. Methodology for molecular engineering and expression of polypeptides in insect cells is described, for example, in Summers and Smith. 1986. A Manual of Methods for Baculovirus Vectors and Insect Culture Procedures, Texas Agricultural Experimental Station Bull. No. 7555, College Station, Tex.; Luckow. 1991. In Prokop et al., Cloning and Expression of Heterologous Genes in Insect Cells with Baculovirus Vectors' Recombinant DNA Technology and Applications, 97-152; King, L. A. and R. D. Possee, 1992, The baculovirus expression system, Chapman and Hall, United Kingdom; O'Reilly, D. R., L. K. Miller, V. A. Luckow, 1992, Baculovirus Expression Vectors: A Laboratory Manual, New York; W. H. Freeman and Richardson, C. D., 1995, Baculovirus Expression Protocols, Methods in Molecular Biology, volume 39; U.S. Pat. No. 4,745,051; US2003148506; and WO 03/074714. A particularly suitable promoter for transcription of the nucleotide sequence of the invention encoding of the parvoviral capsid proteins is e.g. the polyhedron promoter. However, other promoters that are active in insect cells are known in the art, e.g. the p10, p35 or IE-1 promoters and further promoters described in the above references.

Preferably the nucleic acid construct for expression of the parvoviral capsid proteins in insect cells is an insect cell-compatible vector. An “insect cell-compatible vector” or “vector” is understood to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In a preferred embodiment, the vector is a baculovirus, a viral vector, or a plasmid. In a more preferred embodiment, the vector is a baculovirus, i.e. the construct is a baculoviral vector. Baculoviral vectors and methods for their use are described in the above cited references on molecular engineering of insect cells.

The invention thus also relates to a mammalian or insect cell comprising a nucleic acid construct comprising a nucleotide sequence of the invention which is operably linked to expression control sequences for expression in a mammalian or insect cell.

In a preferred embodiment the invention relates to an insect cell comprising a nucleic acid construct of the invention as defined above. Any insect cell which allows for replication of a parvoviral virion/AAV and which can be maintained in culture can be used in accordance with the present invention. For example, the cell line used can be from Spodoptera frugiperda, drosophila cell lines, or mosquito cell lines, e.g., Aedes albopictus derived cell lines. Preferred insect cells or cell lines are cells from the insect species which are susceptible to baculovirus infection, including e.g. Se301, SeIZD2109, SeUCR1, Sf9, Sf900+, Sf21, BTI-TN-5B1-4, MG-1, Tn368, HzAm1, Ha2302, Hz2E5 and High Five from Invitrogen.

Alternatively, in a preferred embodiment the mammalian cell is ex vivo or in vitro.

In a preferred embodiment the mammalian or insect cell of the invention further comprises: (a) a second nucleotide sequence comprising at least one parvoviral inverted terminal repeat (ITR) nucleotide sequence; and (b) a third nucleotide sequence comprising a Rep52 or a Rep40 coding sequence operably linked to expression control sequences for expression in the cell; and (c) a fourth nucleotide sequence comprising a Rep78 or a Rep68 coding sequence operably linked to expression control sequences for expression in the cell.

In the context of the invention “at least one parvoviral ITR nucleotide sequence” is understood to mean a palindromic sequence, comprising mostly complementary, symmetrically arranged sequences also referred to as “A,” “B,” and “C” regions. The ITR functions as an origin of replication, a site having a “cis” role in replication, i.e., being a recognition site for trans acting replication proteins (e.g., Rep 78 or Rep68) which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus replicating in a mammalian cell typically has two ITR sequences. It is, however, possible to engineer an ITR so that binding sites are on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. In view of the safety of viral vectors it may be desirable to construct a viral vector that is unable to further propagate after initial introduction into a cell. Such a safety mechanism for limiting undesirable vector propagation in a recipient may be provided by using recombinant parvovirus with a chimeric ITR as described in US2003148506.

The number of vectors or nucleic acid constructs employed is not limiting of the invention. For example, one, two, three, four, five, six, or more vectors can be employed to produce parvovirus in insect cells in accordance with the present inventive method. If six vectors are employed, one vector encodes parvoviral VP 1, another vector encodes parvoviral VP2, yet another vector encodes parvoviral VP3, still yet another vector encodes Rep52 or Rep40, while Rep78 or Rep 68 is encoded by another vector and a final vector comprises at least one parvoviral ITR. Additional vectors might be employed to express, for example, Rep52 and Rep40, and Rep78 and Rep 68. If fewer than six vectors are used, the vectors can comprise various combinations of the at least one parvoviral ITR and the VP1, VP2, VP3, Rep52/Rep40, and Rep78/Rep68 coding sequences. Preferably, two vectors or three vectors are used, with two vectors being more preferred as described above. If two vectors are used, preferably the insect cell comprises: (a) a first nucleic acid construct for expression of the parvoviral capsid proteins as defined above, which construct further comprises the third and fourth nucleotide sequences as defined in (b) and (c) above, the third nucleotide sequence comprising a Rep52 or a Rep40 coding sequence operably linked to at least one expression control sequence for expression in an insect cell, and the fourth nucleotide sequence comprising a Rep78 or a Rep68 coding sequence operably linked to at least one expression control sequence for expression in an insect cell; and (b) a second nucleic acid construct comprising the second nucleotide sequence as defined in (a) above, comprising at least one parvoviral ITR nucleotide sequence. If three vectors are used, preferably the same configuration as used for two vectors is used except that separate vectors are used for expression of the capsid proteins and for expression of the Rep52, Rep40 Rep78 and Rep68 proteins. The sequences on each vector can be in any order relative to each other. For example, if one vector comprises ITRs and an open reading frame (ORF) comprising nucleotide sequences encoding VP capsid proteins, the VP ORF can be located on the vector such that, upon replication of the DNA between ITR sequences, the VP ORF is replicated or not replicated. For another example, the Rep coding sequences and/or the ORF comprising nucleotide sequences encoding VP capsid proteins can be in any order on a vector. It is understood that also the second, third and further nucleic acid construct(s) preferably are an insect cell-compatible vectors, preferably a baculoviral vectors as described above. Alternatively, in the insect cell of the invention, one or more of the first nucleotide sequence, second nucleotide sequence, third nucleotide sequence, and fourth nucleotide sequence and optional further nucleotide sequences may be stably integrated in the genome of the insect cell. One of ordinary skill in the art knows how to stably introduce a nucleotide sequence into the insect genome and how to identify a cell having such a nucleotide sequence in the genome. The incorporation into the genome may be aided by, for example, the use of a vector comprising nucleotide sequences highly homologous to regions of the insect genome. The use of specific sequences, such as transposons, is another way to introduce a nucleotide sequence into a genome.

In a preferred embodiment of the invention, the second nucleotide sequence present in the insect cells of the invention, i.e. the sequence comprising at least one parvoviral ITR, further comprises at least one nucleotide sequence encoding a gene product of interest, whereby preferably the at least one nucleotide sequence encoding a gene product of interest becomes incorporated into the genome of an parvovirus produced in the insect cell. Preferably, at least one nucleotide sequence encoding a gene product of interest is a sequence for expression in a mammalian cell. Preferably, the second nucleotide sequence comprises two parvoviral ITR nucleotide sequences and wherein the at least one nucleotide sequence encoding a gene product of interest is located between the two parvoviral ITR nucleotide sequences. Preferably, the nucleotide sequence encoding a gene product of interest (for expression in the mammalian cell) will be incorporated into the parvoviral genome produced in the insect cell if it is located between two regular ITRs, or is located on either side of an ITR engineered with two D regions.

The second nucleotide sequence defined herein above may thus comprise a nucleotide sequence encoding at least one “gene product of interest” for expression in a mammalian cell, located such that it will be incorporated into an parvoviral genome replicated in the insect cell. Any nucleotide sequence can be incorporated for later expression in a mammalian cell transfected with the parvovirus produced in accordance with the present invention. The nucleotide sequence may e.g. encode a protein it may express an RNAi agent, i.e. an RNA molecule that is capable of RNA interference such as e.g. a shRNA (short hairpinRNA) or an siRNA (short interfering RNA). “siRNA” means a small interfering RNA that is a short-length double-stranded RNA that are not toxic in mammalian cells (Elbashir et al., 2001, Nature 411: 494-98; Caplen et al., 2001, Proc. Natl. Acad. Sci. USA 98: 9742-47). In a preferred embodiment, the second nucleotide sequence may comprise two nucleotide sequences and each encodes one gene product of interest for expression in a mammalian cell. Each of the two nucleotide sequences encoding a product of interest is located such that it will be incorporated into a recombinant parvovirus genome replicated in the insect cell.

The product of interest for expression in a mammalian cell may be a therapeutic gene product. A therapeutic gene product can be a polypeptide, or an RNA molecule (siRNA), or other gene product that, when expressed in a target cell, provides a desired therapeutic effect such as e.g. ablation of an undesired activity, e.g. the ablation of an infected cell, or the complementation of a genetic defect, e.g. causing a deficiency in an enzymatic activity. Examples of therapeutic polypeptide gene products include CFTR, Factor IX, Lipoprotein lipase (LPL, preferably LPL S447X; see WO 01/00220), Apolipoprotein A1, Porphobilinogen deaminase, Alanine:glyoxylate aminotransferase, Uridine Diphosphate Glucuronosyltransferase (UGT), Retinitis Pigmentosa GTPase Regulator Interacting Protein (RP-GRIP), and cytokines or interleukins like e.g. IL-10.

Alternatively, or in addition as a second gene product, a second nucleotide sequence defined herein above may comprise a nucleotide sequence encoding a polypeptide that serve as marker proteins to assess cell transformation and expression. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3^{rd} edition), Cold Spring Harbor Laboratory, Cold Spring Harbor Laboratory Press, New York. Furthermore, second nucleotide sequence defined herein above may comprise a nucleotide sequence encoding a polypeptide that may serve as a fail-safe mechanism that allows to cure a subject from cells transduced with the recombinant parvoviral virion of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31: 844-849).

In one embodiment, the second nucleotide sequence further comprises at least one nucleotide sequence encoding a gene product of interest (for expression in a mammalian cell) and whereby the at least one nucleotide sequence encoding a gene product of interest becomes incorporated into the genome of an parvoviral virion produced in the cell.

In the recombinant parvoviral vectors of the invention the at least one nucleotide sequence(s) encoding a gene product of interest for expression in a mammalian cell, preferably is/are operably linked to at least one mammalian cell-compatible expression control sequence, e.g., a promoter. Many such promoters are known in the art (see Sambrook and Russel, 2001, supra). Constitutive promoters that are broadly expressed in many cell-types, such as the CMV promoter may be used. However, more preferred will be promoters that are inducible, tissue-specific, cell-type-specific, or cell cycle-specific. For example, for liver-specific expression a promoter may be selected from an α1-anti-trypsin promoter, a thyroid hormone-binding globulin promoter, an albumin promoter, LPS (thyroxine-binding globlin) promoter, HCR-ApoCII hybrid promoter, HCR-hAAT hybrid promoter and an apolipoprotein E promoter. Other examples include the E2F promoter for tumor-selective, and, in particular, neurological cell tumor-selective expression (Parr et al., 1997, Nat. Med. 3:1145-9) or the IL-2 promoter for use in mononuclear blood cells (Hagenbaugh et al., 1997, J Exp Med; 185: 2101-10).

AAV is able to infect a number of mammalian cells. See, e.g., Tratschin et al., Mol. Cell. Biol., 5(11):3251-3260 (1985) and Grimm et al., Hum. Gene Ther., 10(15):2445-2450 (1999). However, AAV transduction of human synovial fibroblasts is significantly more efficient than in similar murine cells, Jennings et al., Arthritis Res, 3:1 (2001), and the cellular tropicity of AAV differs among serotypes. See, e.g., Davidson et al., Proc. Natl. Acad. Sci. USA, 97(7):3428-3432 (2000) (discussing differences among AAV2, AAV4, and AAV5 with respect to mammalian CNS cell tropism and transduction efficiency).

Parvoviral sequences that may be used in the present invention for the production of parvoviral virions in insect cells can be derived from the genome of any AAV serotype as has been defined above or can be newly developed parvoviral sequences e.g., by directed evolution, by shuffling or by rational design. Preferred parvoviral sequences that may be used in the present invention will be further discussed hereafter.

Preferably the parvoviral ITR sequences for use in the context of the present invention are derived from AAV1, AAV2, and/or AAV4. Likewise, the Rep52, Rep40, Rep78 and/or Rep68 coding sequences are preferably derived from AAV1, AAV2, and/or AAV5. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., 1999, J. Virol., 73(2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells.

The AAV VP proteins are known to determine the cellular tropicity of the AAV virion. The VP protein-encoding sequences are significantly less conserved than Rep proteins and genes among different AAV serotypes. The ability Rep and ITR sequences to cross-complement corresponding sequences of other serotypes allows for the production of pseudotyped AAV particles comprising the capsid proteins of a serotype (e.g., AAV3) and the Rep and/or ITR sequences of another AAV serotype (e.g., AAV2). Such pseudotyped AAV particles are a part of the present invention.

Modified “parvoviral” sequences also can be used in the context of the present invention, e.g. for the production of recombinant parvoviral vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAV5, AAV6, AAV7, AAV8 or AAV9 ITR, Rep, or VP can be used in place of wild-type parvoviral ITR, Rep, or VP sequences.

In a third aspect the invention relates to a parvoviral virion. Preferably the parvoviral virion comprising a capsid protein that comprises at least one immune evasion repeat of the invention as defined above.

In one embodiment at least one immune evasion repeat is present in a VP3 capsid protein. Preferably, an immune evasion repeat is present in at least one position in the parvoviral VP3 capsid protein that is immediately N-terminal to an amino acid in the VP3 capsid protein that corresponds to amino acid position selected from the group consisting of amino acid positions 226, 255, 377, 444, 453, 488, 652, 697 and 726 of the AAV5 capsid protein. More preferably, the immune evasion repeat is present in at least one position in the parvoviral VP3 capsid protein that is immediately N-terminal to an amino acid in the VP3 capsid protein that corresponds to amino acid position selected from the group consisting of amino acid positions 255, 377, 444, 652, 697 and 726 of the AAV2/5 hybrid capsid protein. Most preferably, immune evasion repeat is present in at least one position in the parvoviral VP3 capsid protein that is immediately N-terminal to an amino acid in the VP3 capsid protein that corresponds to amino acid position selected from the group consisting of amino acid positions 255, 377 and 444 of the AAV2/5 hybrid capsid protein, of which amino acid positions 255 is most preferred.

Preferably, the parvoviral virion comprises in its genome at least one nucleotide sequence encoding a gene product of interest, whereby the at least one nucleotide sequence is not a native parvoviral nucleotide sequence, and whereby in the stoichiometry of the parvoviral VP1, VP2, and VP3 capsid proteins the amount of VP1: (a) is at least 100, 105, 110, 120, 150, 200 or 400% of the amount of VP2; or (b) is at least 8, 10, 10.5, 11, 12, 15, 20 or 40% of the amount of VP3; or (c) is at least as defined in both (a) and (b). Preferably, the amount of VP1, VP2 and VP3 is determined using an antibody recognizing an epitope that is common to each of VP1, VP2 and VP3. Various immunoassays are available in the art that will allow quantify the relative amounts of VP1, VP2 and/or VP3 (see e.g. Using Antibodies, E. Harlow and D. Lane, 1999, Cold Spring Harbor Laboratory Press, New York). An suitable antibody recognizing an epitope that is common to each of the three capsid proteins is e.g. the mouse anti-Cap B1 antibody (as is commercially available from Progen, Germany).

In another aspect, the invention relates to a capsid protein comprising an immune evasion repeat, preferably in a VP3 capsid protein as described above.

In another aspect the invention relates to a parvoviral virion of the invention for use as a medicament.

Delivery of the parvoviral virion may be via any administration route, preferably via a parental route e.g., injection or infusion by subcutaneous, intravenous, intraperitoneal, intramuscular, intra-arterial or intralesional routes. Administration may alternatively be performed by isolated limb perfusion or variants thereof (U.S. Pat. No. 6,177,403) or by administration to the central nervous system (CNS), e.g. by injection into the ventricular region, striatum, spinal cord and neuromuscular junction, cerebellar lobule with a needle, catheter or related device using neurosurgical techniques known in the art (e.g. Stein et al., J. Viol 73:3424-3429, 1999; Davidson et al., PNAS 97:3428-3432, 2000; Davidson et al., nat. Genet. 3:219-223, 1993; and Alisky and Davidson, Hum. Gene Ther. 11:2315-2329, 2000).

In another aspect the invention relates to a parvoviral virion of the invention for use in the treatment of a subject with pre-existing immunity, for example T cell immunity or the presence of neutralising antibodies, against the parvoviral virion. Preferably the treatment comprises or consists of gene therapy. Such gene therapy can be useful for the treatment or prevention of disease states as indicated below. Gene therapy according to the invention may be useful where readministration and/or repeated administration is required. That is to say, the invention may be especially useful in the treatment of a subject, wherein the subject receives administration of a parvoviral virion on more than one occasion, for example two times, three times, four times, five times or more.

The term “pre-existing T cell immunity” herein refers to memory cytotoxic T cells or cytotoxic T-cells that are present in the subject due to a previous contact or infection with a parvovirus or a recombinant parvoviral gene therapy vector. The previous contact may or may not be with a parvovirus or vector having capsids of that particular type, e.g. that particular AAV serotype. Infection of humans by a variety of AAV serotypes may occur at any stage during life time, including childhood or possibly even in utero. In addition pre-existing T cell immunity may be the consequence of previous administration(s) of recombinant parvoviral gene therapy vectors. Such pre-existing T cell immunity may compromise the efficacy of parvoviral virions in gene therapy, which problems the present invention aims to circumvent.

The invention embraces the delivery of parvoviral virions comprising a nucleotide sequence encoding for a gene of interest, which are useful for the treatment or prevention of disease states in a mammalian subject. Such disease states include, but are not limited to: glycogen storage deficiency type 1A; Pepck deficiency; galactosemia; phenylketonureia; Maple syrup urine disease; tyrosinemia type 1; methylmalonic acidemia; medium chain acetyl CoA deficiency; ornithine transcarbamylase deficiency; citrullinemia; familial hypercholesterolemia; Crigler-Najjar disease; severe combined immunodeficiency disease; Gout and Lesch-Nyan syndrome; biotinidase deficiency; Gaucher disease; Sly syndrome; Zellweger syndrome; acute intermittent porphyria; hyperoxaluria (type 1); alpha-1 antitrypsin deficiency (emphysema); anemia due to thalassemia or to renal failure; ischemic diseases; occluded blood vessels as seen in e.g., atherosclerosis, thrombosis or embolisms; Parkinson's disease; congestive heart failure; various cancers; inflammatory and immune disorders; muscular dystrophies; diabetes; hemophilia A; hemophilia B; Factor VII deficiency; Factor X deficiency; Factor XI deficiency; Factor XIII deficiency; Protein C deficiency; ApoA-1 deficiency and LPL-responsive conditions selected from the group consisting of: complete LPL deficiency, type 1 hyperlipoproteinemia, type 5 hyperlipoproteinemia, chylomicronemia hyperlipidemia, partial LPL deficiency, pancreatitis, hypertriglyceridemia, hypoalphalipoproteinemia (low HDL-cholesterol), cardiovascular disease, coronary heart disease, coronary artery disease, atherosclerosis, angina pectoris, hypertension, cerebrovascular disease, coronary restenosis, peripheral vascular disease, diabetes, cachexia and obesity.

In another aspect the invention relates to a pharmaceutical composition comprising a parvoviral virion of the invention and a pharmaceutically acceptable carrier.

Also, the invention relates to a method of gene therapy, wherein the method comprises the step of administering an effective amount of a parvoviral virion as defined herein to a subject in need thereof. The method may be carried out such that more than one administration of a parvoviral virion is carried out.

A pharmaceutical carrier can be any compatible, non-toxic substance suitable to deliver the active ingredients, i.e. the parvoviral virion of the invention, to a patient. Sterile water, alcohol, fats, waxes, and inert solids may be used as the carrier. Preparations for parental administration must be sterile. A pharmaceutical composition of the invention may be delivered via an administration route as described above.

Also, the invention relates to a method for producing an parvoviral virion, comprising the steps of: (a) culturing a mammalian or insect cell of the invention under conditions such that the parvoviral virion is produced; and, (b) recovery of the parvoviral virion. Growing conditions for insect cells in culture, and production of heterologous products in insect cells in culture are well-known in the art and described e.g. in the above cited references on molecular engineering of insects cells.

Preferably the method of the invention further comprises the step of affinity purification of the parvoviral virion using an anti-parvoviral antibody, preferably an immobilized antibody. The anti-parvoviral antibody preferably is an monoclonal antibody. A particularly suitable antibody is a single chain cameloid antibody or a fragment thereof as e.g. obtainable from camels or llamas (see e.g. Muyldermans, 2001, Biotechnol. 74: 277-302). The antibody for affinity-purification of parvoviral virions preferably is an antibody that specifically binds an epitope on a parvoviral capsid protein, whereby preferably the epitope is an epitope that is present on capsid protein of more than type of parvovirus, e.g. on more than one AAV serotype. E.g. the antibody may be raised or selected on the basis of specific binding to AAV2 capsid but at the same time also it may also specifically bind to AAV 1, AAV3 and AAV5 capsids.

Furthermore, the invention relates to a method for treating a subject suffering from a disease that may be treated using gene therapy with a parvoviral virion of the invention or with a pharmaceutical composition of the invention to reduce T-cell mediated destruction of cells and/or inhibition by neutralising antibodies that are infected with the parvoviral virion as compared to a parvoviral virion comprising a capsid protein without a minimal GAr region. Preferably, the amount of the parvoviral virion or the pharmaceutical composition is sufficient to express the protein of interest at a level that provides a therapeutic effect.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### DESCRIPTION OF THE FIGURES

FIG. 1: Alignment of the nucleotide sequences of several AAV serotypes. The following sequences have been aligned (GenBank accession numbers in brackets): AAV2/5 (pFBDvp256); AAV1_(NPO49542); AAV2_(NC001401); AAV3_(NPO43941); AAV4_(NPO44927); AAV5_(YP068409); AAV6_(AF028704); AAV7_(YP077178); AAV8_(YP077180); AAV9_(AY530579); AAV10_(AY631965); AAV11_(AY631966); AAV12_(DQ813647); AAVbovine_(YP024971); AAV DA1_(YP077183); AAV VR-865_(NP852781); AAV-Go.1_(AY724675); AAV VR-355_(DQ180605); Rat AAV1_(DQ100363); Mouse AAV1_(DQ100362); AAV hu.49_(AY530612); AAV hu.34_(AY530598); AAV hu.21_(AY530587); AAV pi.2_AY530554.

FIG. 2: Site directed mutagenesis PCR according to the Stratagene QuikChange® XL kit. (1.) A mutant strand is synthesized, whereby thermal cycling is performed to denature DNA template, anneal the mutagenic primers containing the desired mutation and to extend and incorporate primers with a high fidelity polymerase (PfuUltra DNA polymerase). A PCR with sense/antisense primers is run using a high fidelity polymerase. The entire plasmid is including the mutation is amplified during each round of amplification. (2.) After the PCR reaction, parental hemimethylated DNA is digested by DpnI and (3.) transformed into competent cells for nick repair. Plasmids will need to be screened by DNA sequencing to select clones that contain insertions.

FIG. 3: Gateway recombination of pDonr221-GAr into the baculo expression vector (pvd166). Sequences flanked by att sites in pDonr221-GAr recombine to the att sites of the GAr expression vector (pvd166) in the presence of LR clonase. The result of this recombination reaction is a Cap2/5GAr gene between the att sites of the GAr expression vector and a byproduct of the LR reaction. When transformed to competent cells and grown in the presence of ampicilin only the Cap2/5GAr vector is able to amplify. Byproduct of the recombination reaction will fail to amplify, because of the ccdb and presence of kanamycine on the vector.

FIG. 4: Restriction digests of final GAr vectors. Gel 1 shows a restriction digest with DraIII and EcoRI, which will result in a linearized vector when recombination has taken place and 4371 and 3417 bp fragments when recombination failed. Gel 2 shows a restriction digest with RsrII and SnaBI which will result in two fragments of 6091 and 2112 bp. The faint band above the 6 kb band in gel 2 is due to poor digestion by one of enzymes. Numbers above the lanes represent pvd numbers of the GAr vectors.

FIG. 5: FlashBAC™ recombination to produce Cap2/5GAr expressing baculoviruses. FlashBAC™ backbone is transfected together with one of the Cap2/5GAr baculo expression vectors. Homologous recombination between the two ORFs present on the FlashBAC™ backbone and Cap2/5GAr expression vector restores function of the essential gene present on the baculovirus backbone. This results in a baculovirus that is able to replicate and that is expressing Cap2/5GAr.

FIG. 6: LPL mass infectivity results. 84-31 cells were infected with 5 purified AAV2/5GAr stocks with LPL at an multiplicity of infection (MOI) of 1000 and of 10,000. Supernatant was harvested 24 h post infection and assayed for total LPL mass by an ELISA kit. In the graph the concentration of LPL (ng/ml) is plotted vs the different constructs.

FIG. 7: The inhibition of infection of target cells by AAV5 wt in presence of wild type and GAr construct plasma.

FIG. 8: The inhibition of infection of target cells by AAV5 wt in presence of plasma from wild type and the 2 GAr constructs, G382 and G267.

FIG. 9: The inhibition of infection of target cells with GAr382 in presence of plasma from wild type and the two GAr constructs, G382 and G267.

FIG. 10: The inhibition of infection of target cells with GAr267 in presence of plasma from wild type and the two GAr constructs, G382 and G267.

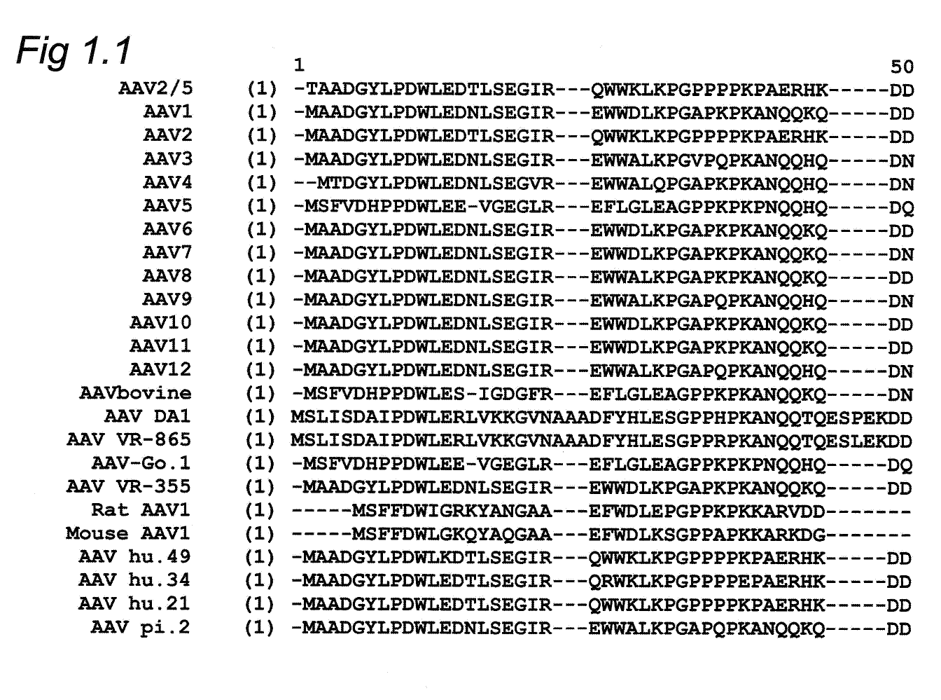

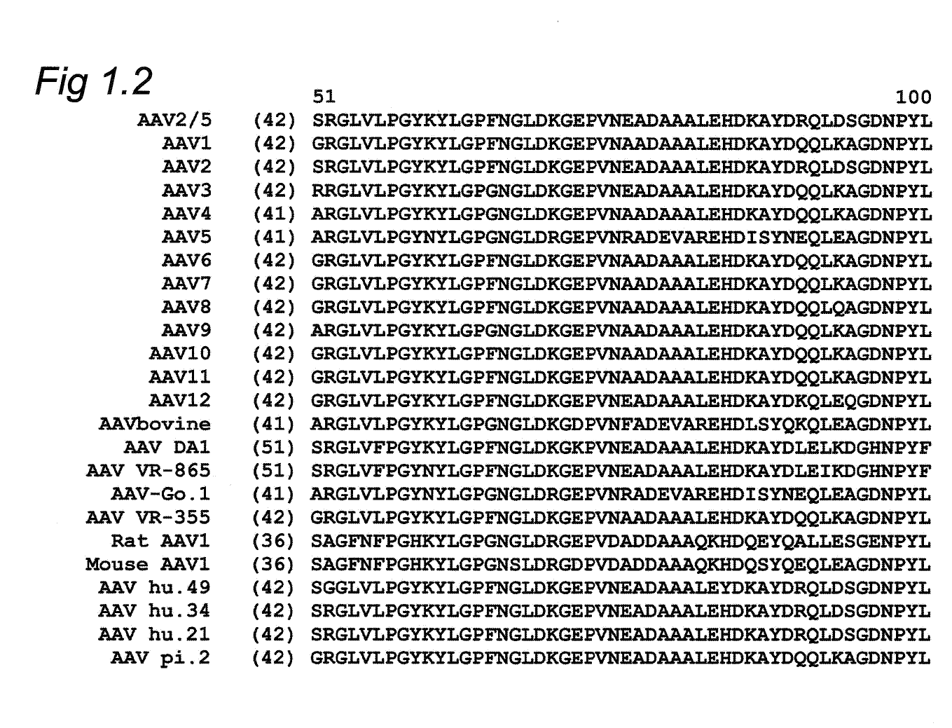

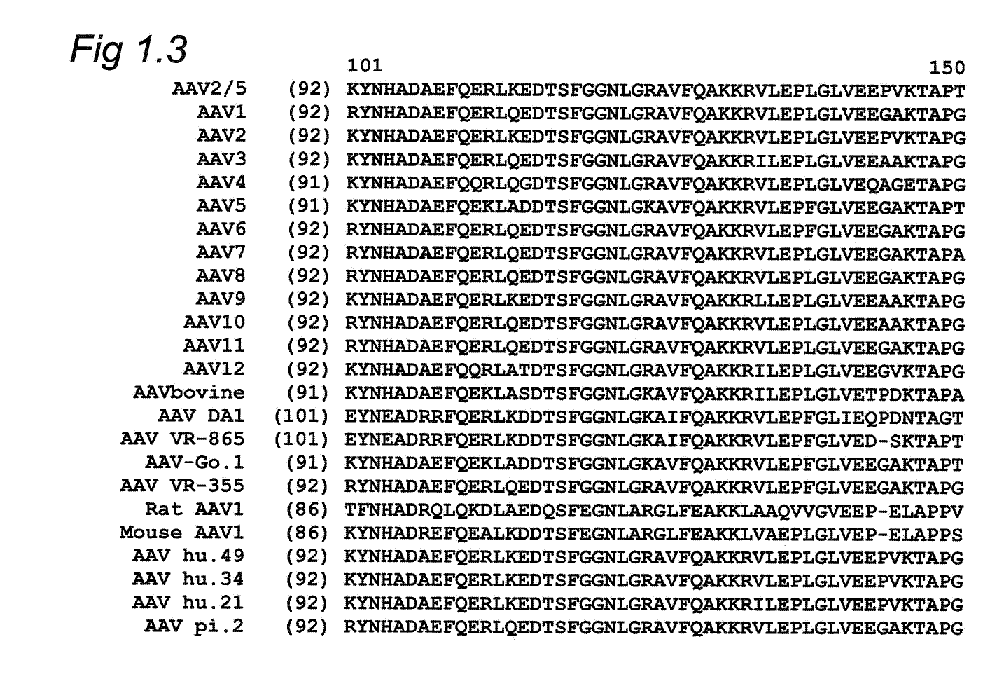

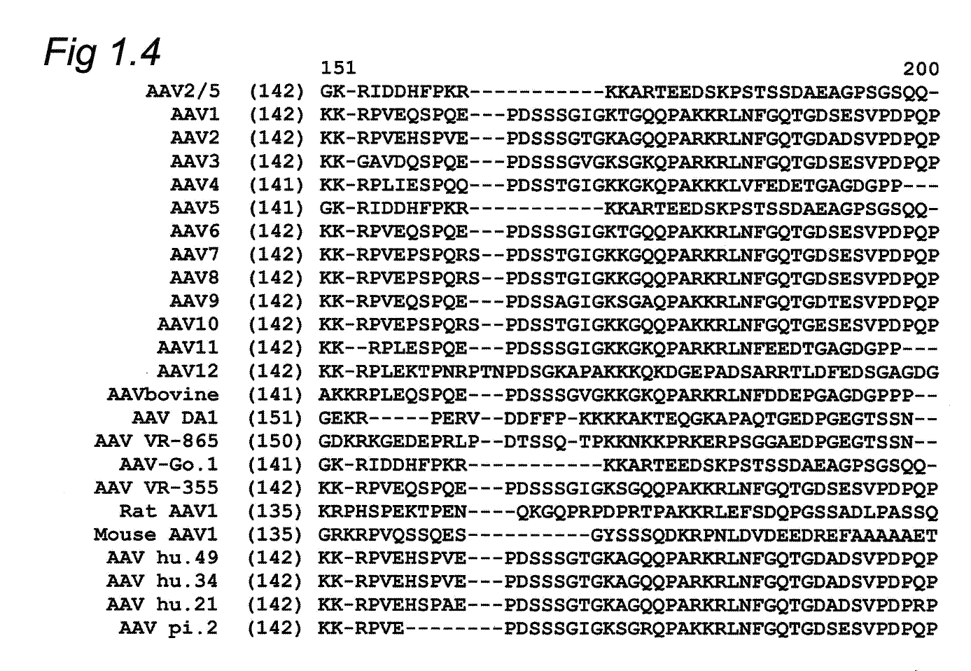

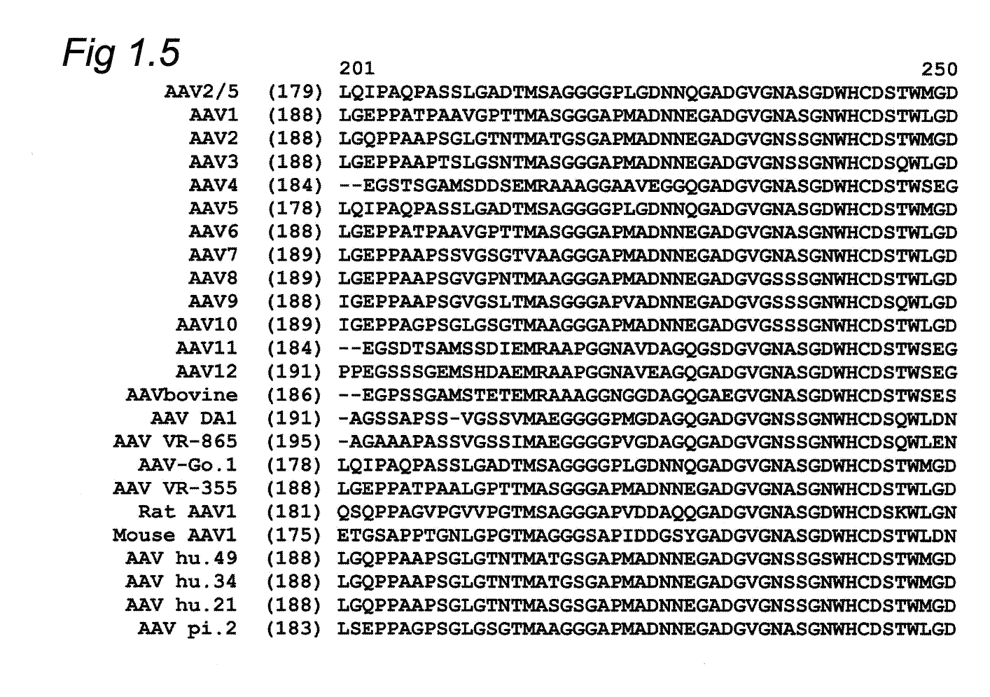

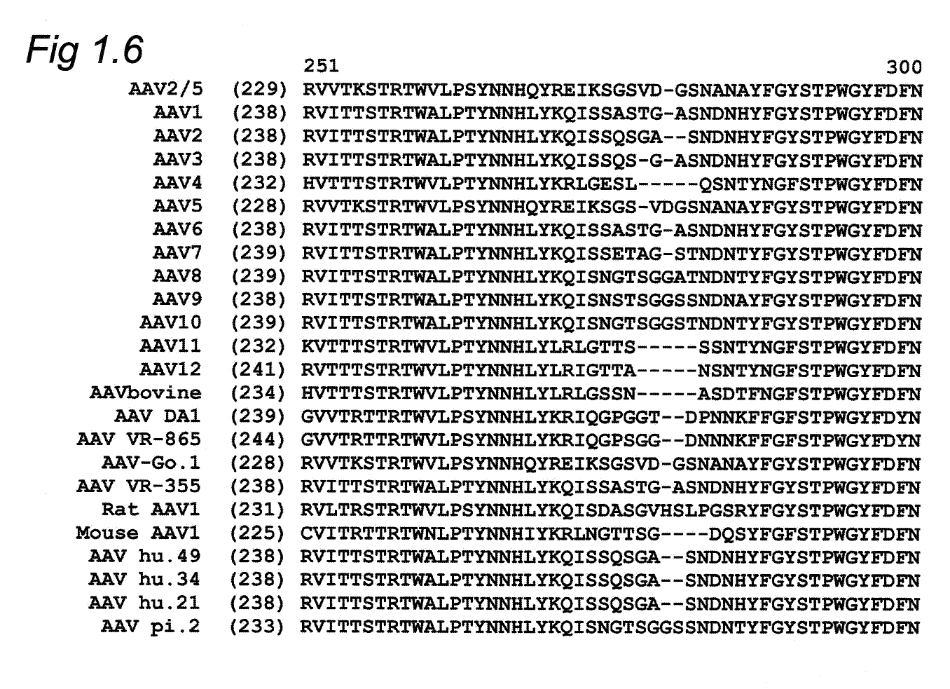

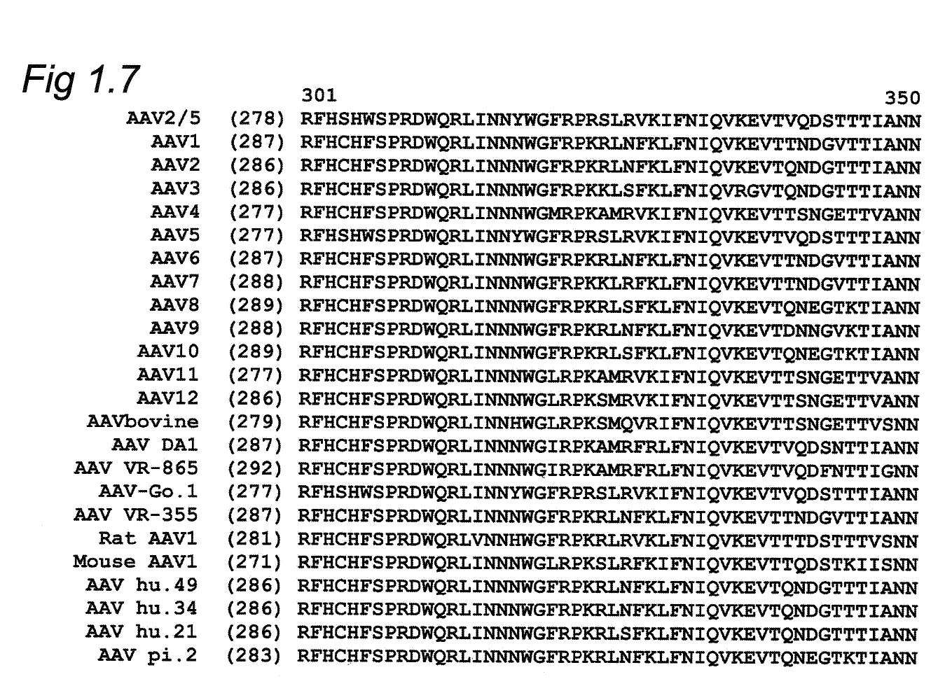

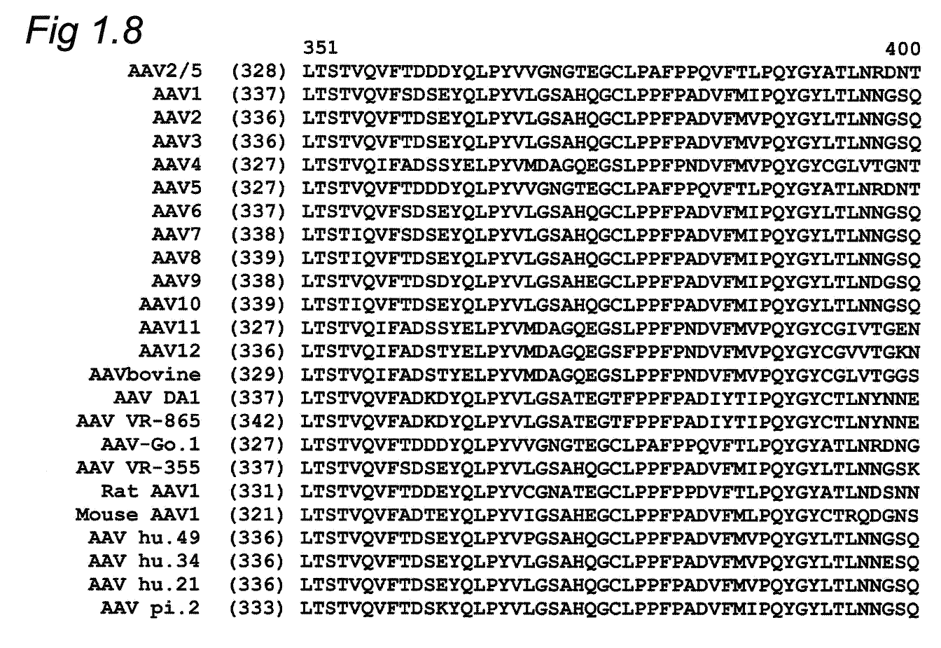

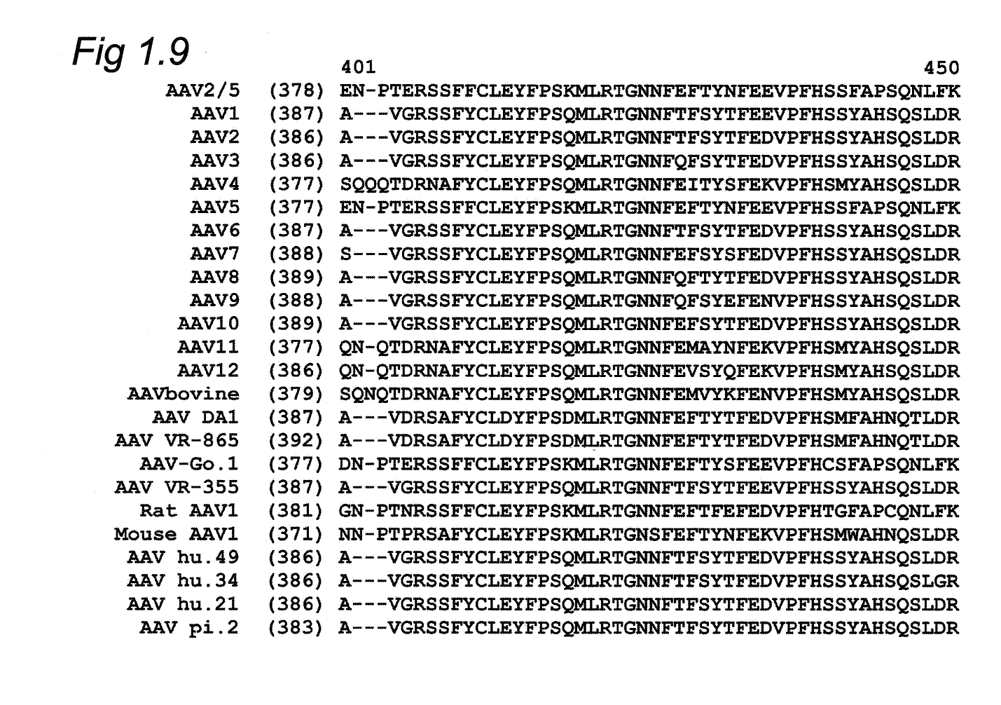

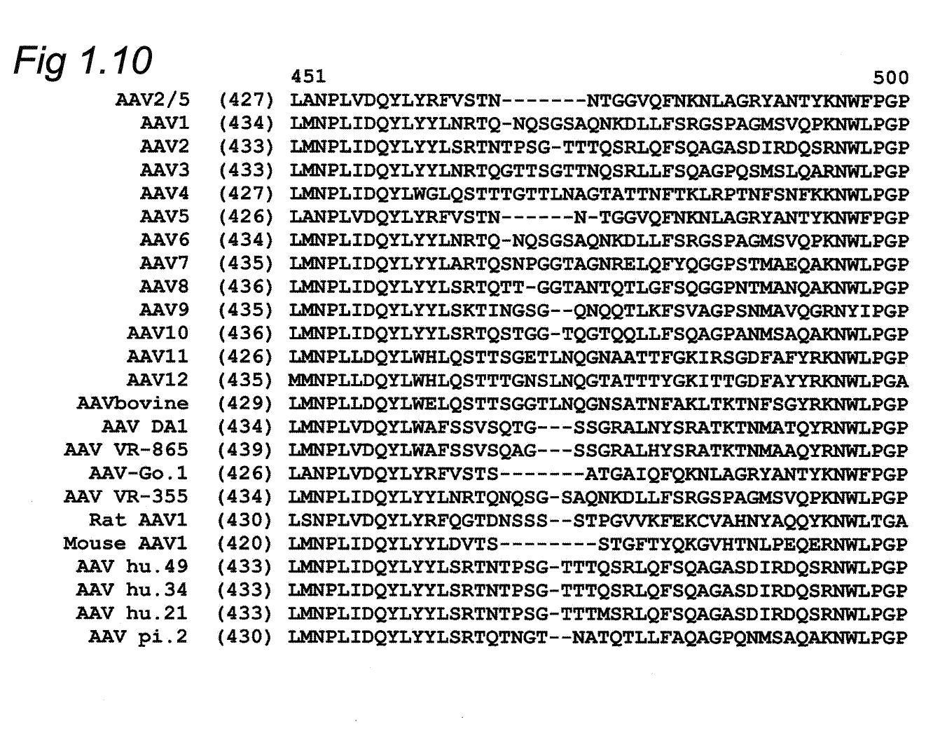

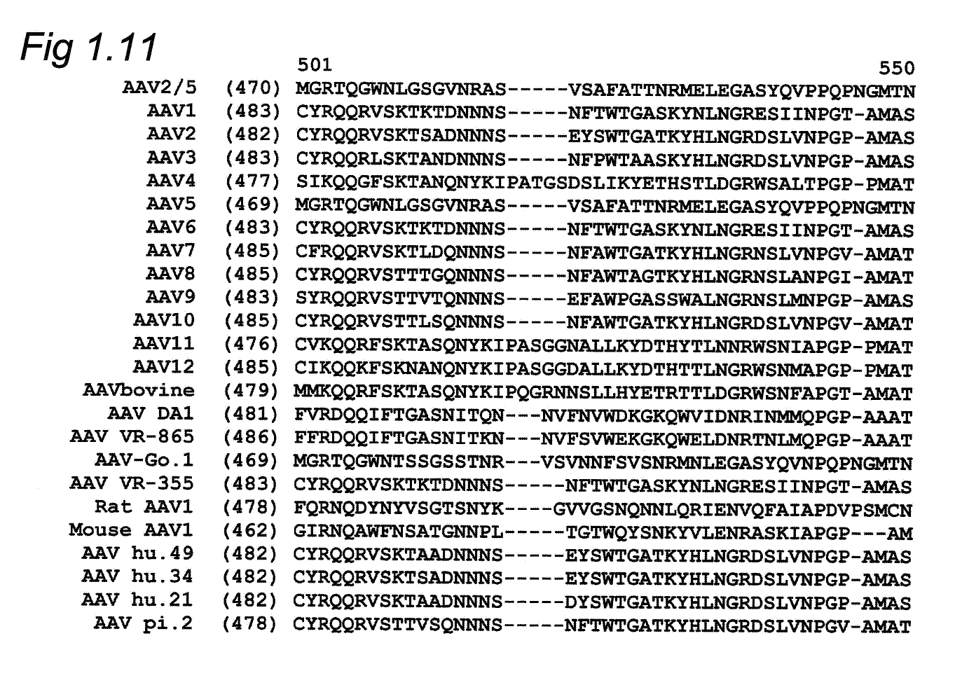

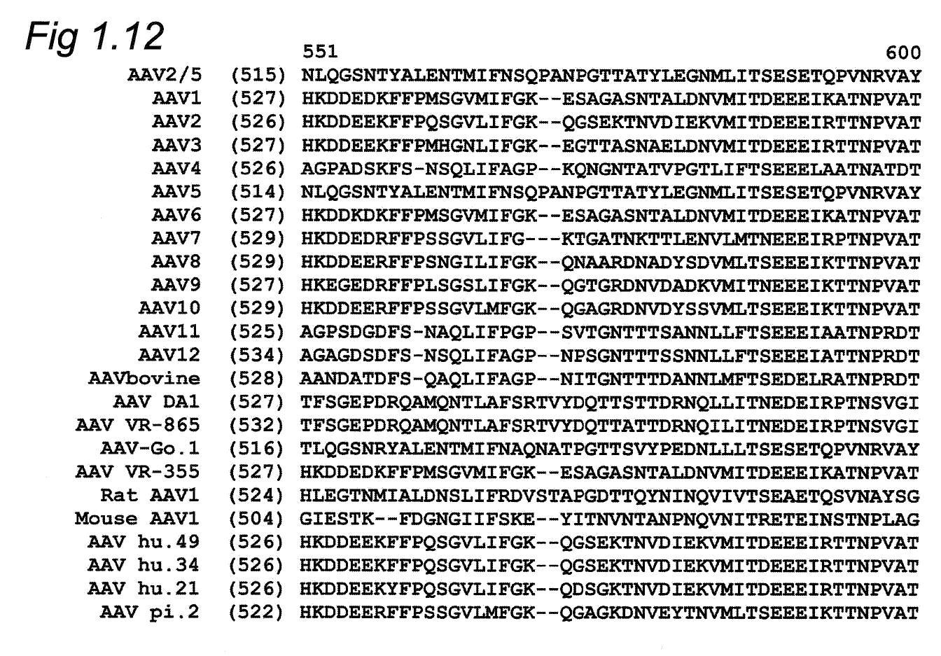

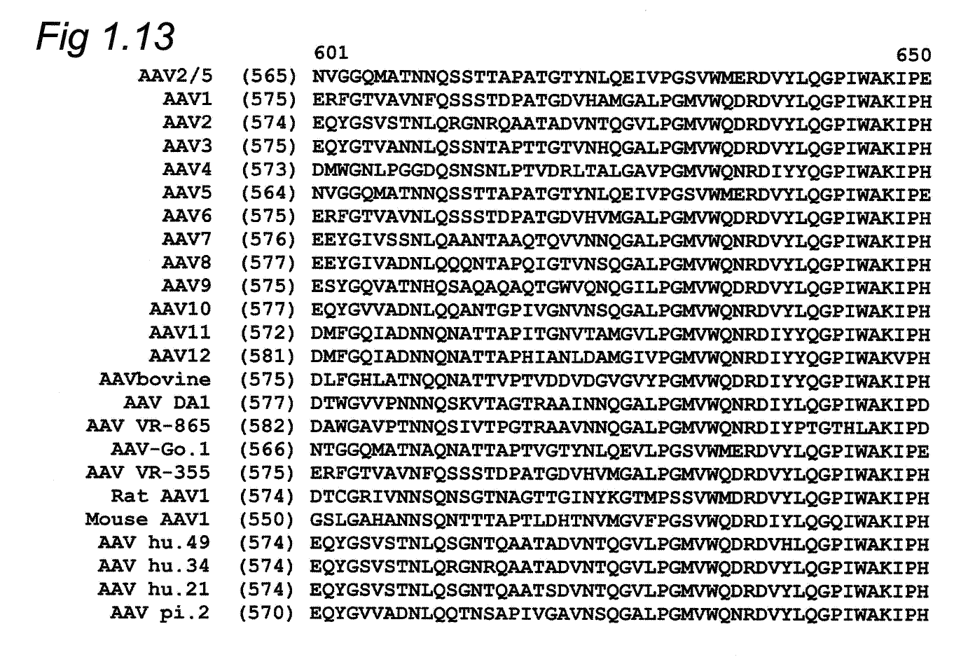

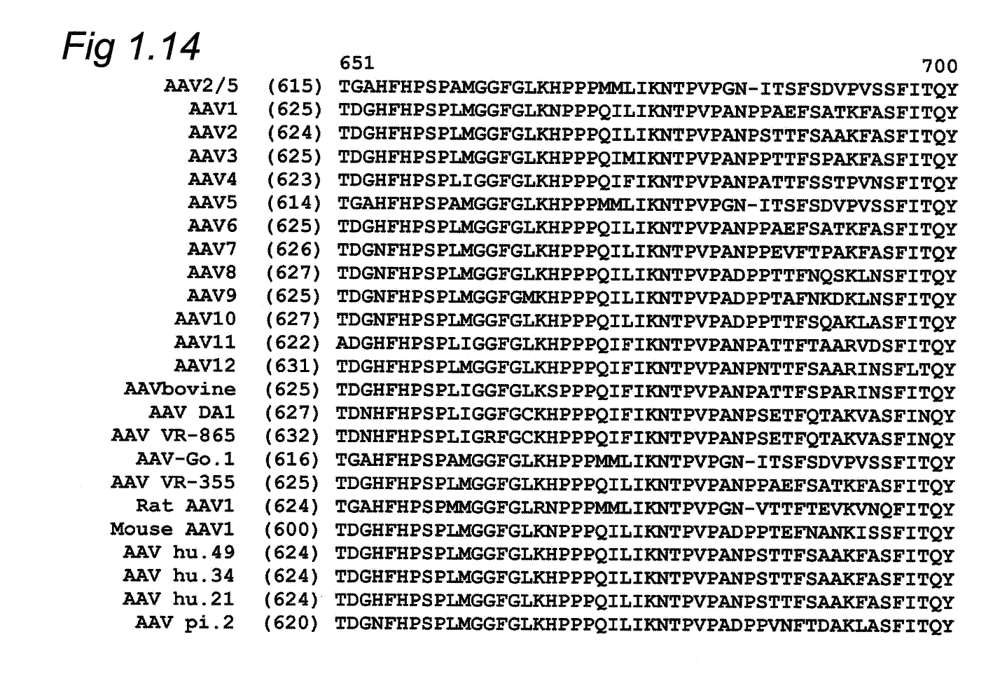

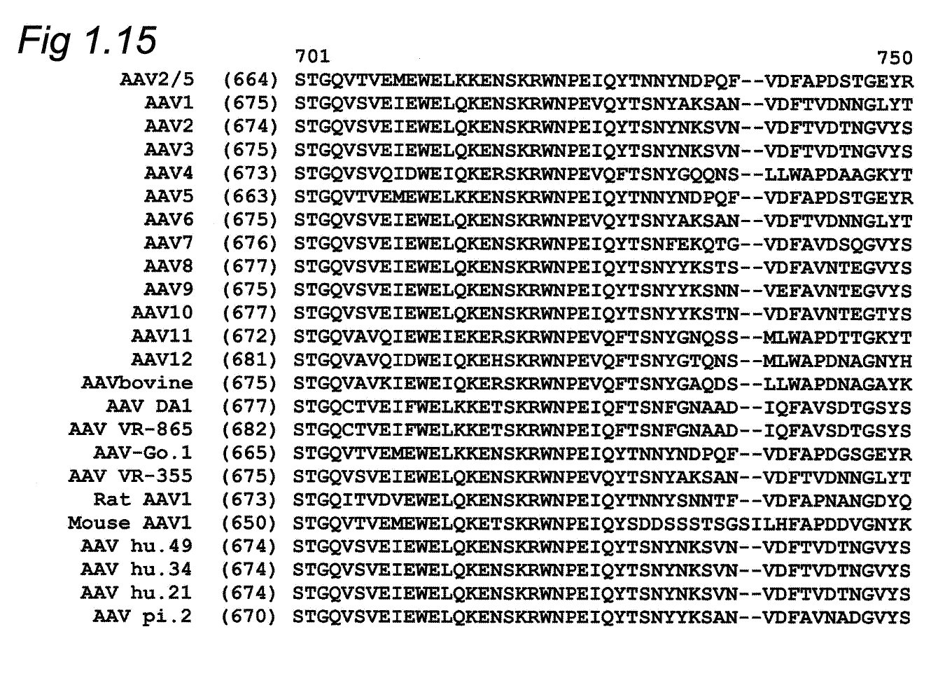

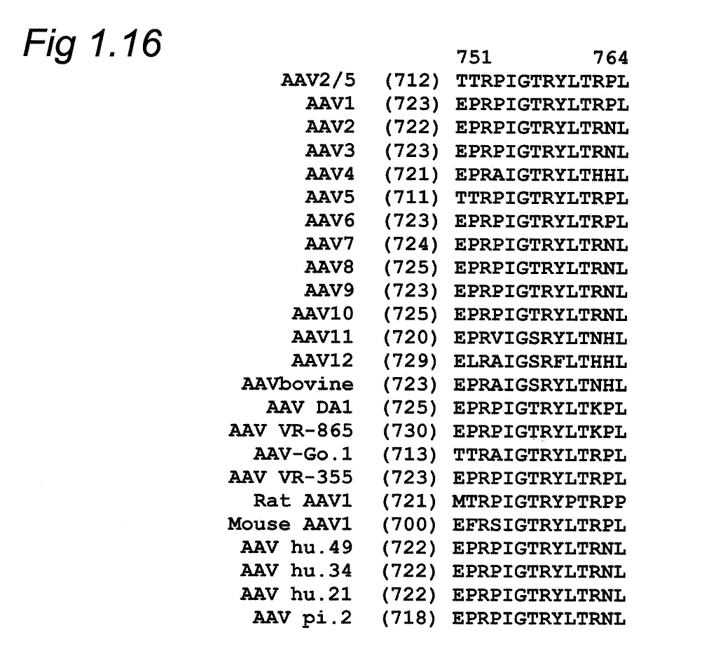

### EXAMPLES

Hereafter the vector construction, baculovirus generation and AAV production of AAV2/5 capsid genes that have a minimal Gly-Ala repeat (GAr) inserted at 13 different locations in the VP3 protein of a AAV2/5 hybrid (Urabe et al. (2006) Journal of Virology 80:1874-1885) is described. Out of the 13 insertion sites in the AAV2/5 capsid selected for GAr insertions, 9 have been made (Table 1). Capsid genes of AAV2/5 comprising a minimal GAr were created by site directed mutagenesis. The nucleic acid sequences and corresponding amino acid sequences of the VP3 capsid comprising minimal GAr insertion are provided in SEQ ID NO:1, 3, 5, 7, 9, 11, 13, 15, and 17 and SEQ ID NO: 2, 4, 6, 8, 10, 12, 14, 16 and 18, respectively. Subsequently, the mutated capsid genes were recombined with a plasmid to produce baculoviruses, which were used to produce AAVs comprising the GAr in their capsid. These AAVs are used to access GAr functionality both in vitro and in vivo. GAr is expected to prevent antigen peptide generation in cis, but not to affect antigen peptide generation in trans, i.e. presentation of antigens derived from other proteins than capsid proteins is not prevented.

TABLE 1
Overview of the insertion places
of the GAr sequence (GGAGAGAG).

| | AAV1 insertion | AAV5 insertion | AAV capsid 2/5 |
|---|---|---|---|
| | place | place | insertion place |
| Cap2/5 - g236 | 235 | 225 | 226 |
| Cap2/5 - g267 | 266 | 254 | 255 |
| Cap2/5 - g382 | 384 | 376 | 377 |
| Cap2/5 - g454 | 451 | 443 | 444 |
| Cap2/5 - g467 | 466 | 452 | 453 |
| Cap2/5 - g502 | 501 | 487 | 488 |
| Cap2/5 - g663 | 663 | 650 | 652 |
| Cap2/5 - g708 | 705 | 696 | 697 |
| Cap2/5 - g751 | 736 | 725 | 726 |

### Example 1

### Vector Construction

### 1.1 Site Directed Mutagenesis PCR

To introduce the GAr sequence into the Cap2/5 gene a site directed mutagenesis approach was used. To create a specific mutation in the capsid gene, the Stratagene QuikChange® XL kit was utilized. This kit can introduce mutations at a specific location when sense and anti-sense primers that contain the GAr sequence are used in a PCR reaction. Primers that insert a GAr sequence into the Cap2/5 gene were designed for 13 different sites in the VP3 protein. These sites have been previously described in literature, were it was shown that insertions at these sites could produce infective AAV particles. Most of these sites are located in AAV2/5 hypervariable regions. Hypervariable regions are stretches of the Cap protein that have the least evolutionary pressure on their protein sequence. Amino acid differences between AAV serotypes are at their highest in these protein stretches. It is hypothesized that AAV would be better able to tolerate an insertion at these locations without losing its infective properties than at locations that are subject to a higher degree of evolutionary conservation.

GAr insertion sites are located both on the outside and inside of the AAV particle when it is packaged into its final form. It is important to have a spread of these insertion sites because most likely not all sites will result infective AAV particles or even packaging. The location of the GAr insert could also be important for its ability to suppress the immune response. It is described in literature that the optimum location for GAr insertions is on the carboxy terminal side of the immune dominant epitope of a protein (U.S. Pat. No. 5,833,991). Table 4 shows the insertion sites of all the used primers. The number represents the location of the GAr insert in the amino acid sequence of the VP3 AAV serotype 5 protein. The GAr sequence in the insertion primers is modelled after the optimal sequence for proteasome inhibition as described by Sharipo et al. (FEBS Lett (2001) 499:137-142): GGAGAGAG (SEQ ID NO: 28).

Primers for the mutagenesis PCR are described in table 2. All insertions are made in a pDonr221 plasmid that contains the Cap2/5 gene. PCR reactions were performed according to the manufacturer's specifications in a Biometra PCR machine. The following reaction mix was used for the PCR: 50 ng pDonr221-Cap2/5 with 125 ng sense and antisense GAr primer, 1 μl dNTP mix, 3 μl Quicksolution, 10 units PfuUltra, 5 μl 10× reaction buffer (from Stratagene QuikChange® XL kit). MilliQ (MQ) was added to a final volume of 50 μl. The PCR is performed using an Ultrahigh fidelity polymerase (PfuUltra) to prevent unwanted mutations. The used PCR program was: 2′ 95° C. initial denaturation, followed by 18 cycles of l′ at 95° C. denaturation, 1′ at 60° C. annealing and 8′ at 68° C. (2′ per kb) elongation was concluded with 8′ at 68° C. This PCR amplifies the entire plasmid including the GAr sequence from the primers. Following amplification, the parental methylated DNA, which does not contain the mutation, is digested with 10u of DpnI (Stratagene) to result in a reaction mix that only contains plasmids that have a GAr sequence in their capsid gene.

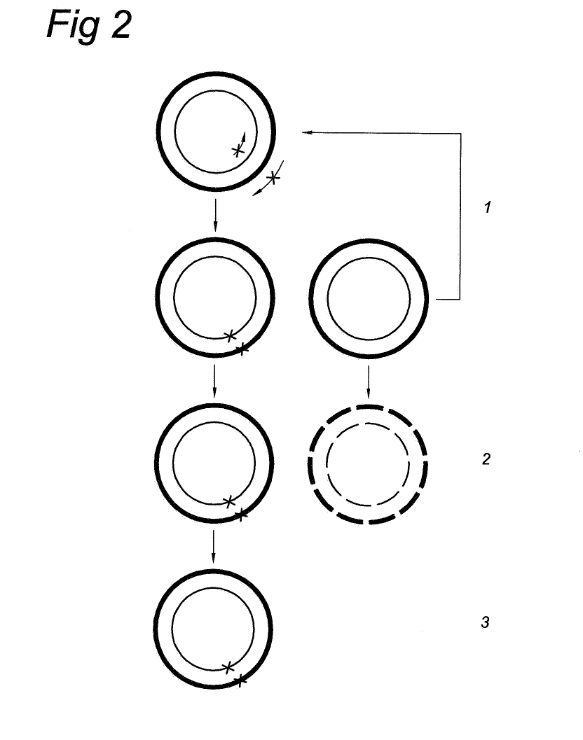

2 μl of digested PCR product was then transformed into XL-10 gold competent cells (Stratagene), to repair nicks that were introduced during the PCR as well as to further amplify the plasmid (FIG. 2), and grown overnight on Kanamycine LB-agar plates 50 μg/ml (Calbiochem). The following day, six clones of each GAr insertion were picked and grown overnight for miniprep DNA isolation (Sigma) in LB-medium supplemented with Kanamycine 50 μg/ml. Out of these six clones, three were send to BaseClear (Leiden, The Netherlands) for DNA sequencing using the primers described in table 3. Sequencing allowed for the identification of clones that had a GAr sequence inserted in their Cap2/5 gene.

DNA sequencing data showed that 9 insertions out of 13 were successfully made. These 9 clones contained the GAr sequence in their capsid gene but no mutations in the capsid gene due to the mutagenesis PCR. Mutagenesis PCR of the 4 remaining insertions was repeated 3 times, which all failed to produce an insertion. It is possible that Cap2/5 template DNA at the location of the 4 remaining sites interfered with primer binding (e.g. high gc template or secondary structure of template DNA) and therefore have prevented insertion of the GAR sequence. Sequence results from the mutagenesis PCR are summarized in SEQ ID NO: 1, 3, 5, 7, 9, 11, 13, 15 and 17.

### 1.2 Gateway Recombination and Vector Control

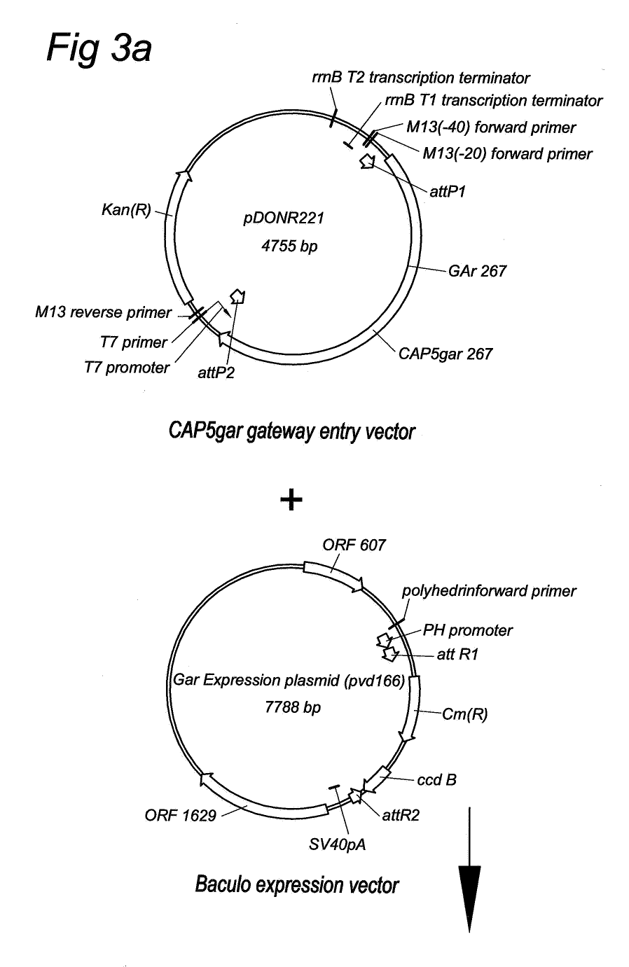

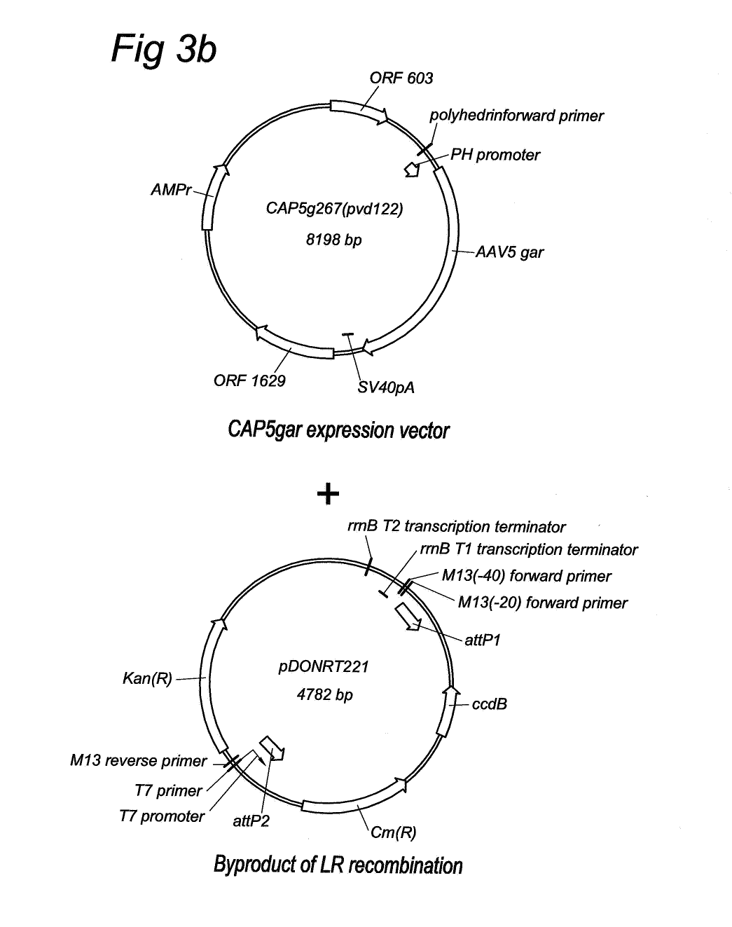

The Cap2/5-GAr vector created using the mutagenesis PCR is by itself not able to express Cap2/5GAr proteins. To be able to express Cap2/5GAr proteins via the baculovirus system the mutated gene needs to be introduced in a vector that can be used for baculovirus expression. Thereto, clones that contained a correct GAr insertion in their capsid gene were used for recombination to a vector that is able to express capsid proteins in the Baculovirus system (pvd166). Pvd166 is a plasmid based on the pAcDB3 plasmid (BD Biosciences, #554825), it comprises a polyhedrin promoter, a gateway cassette from the gateway conversion kit (Invitrogen #11828-029), and a SV40 polyA signal sequence. Recombinations were performed using the Gateway vector conversion system from Invitrogen. This system allows conversions between vectors that have sequences flanked by att recombination sites. A graphic representation of the recombination reaction is given in FIG. 3.

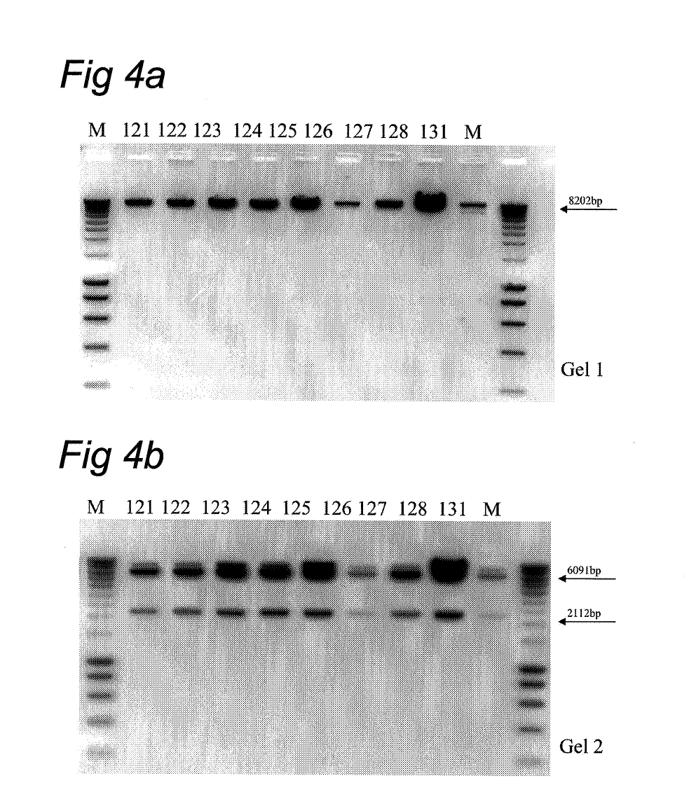

To facilitate recombination, 2 μl of LR clonase II enzyme mix (Invitrogen) was combined with 150 ng of plasmid DNA from the GAr clones, 150 ng of expression vector (pvd166) and brought to a final volume of 10 μl using TE buffer (10 mM Tris-Hcl, 1 mM EDTA, pH=8.0). Reactions were incubated overnight at 25° C. Following overnight incubation 2 μg of Proteinase K (10′ at 37° C., Invitrogen) was added to the reaction to degrade the LR clonase II enzyme mix. Next, recombined DNA was transformed in Xl-10 gold (stratagene) competent cells and grown overnight on LB-agar plates supplemented with 50 μg/ml Ampicilin (Sigma). 6 clones per insertion were screened for the recombination of the GAr-capsid into the expression vector (pvd166). Out of these 6 clones, one was picked and grown in LB-medium supplemented with 50 μg/ml Ampicilin for a maxiprep DNA isolation (Qiagen kit). Maxipreps were screened by restriction digests for the presence of AAV2/5. DNA was digested with DraIII and EcoRI resulting in a linearized vector when recombination has taken place. If recombination has failed, 4371 and 3417 bp fragments will be seen after loading on and running of an agarose gel. DNA was also digested with RsrII and SnaBI resulting in 6091 and 2112 bp fragments when recombination has taken place. Digested DNA is run in a 1% Agarose gel for 1 hour at 100V in a Horizon electrophoresis system (Biometra). Restriction digests from the maxiprep used for Baculo generation are shown in FIG. 4. Constructs have now been generated (Cap2/5GAr) comprising nucleotide sequences that encode for GAr modified Cap proteins, which vector can be used to express the GAr modified Cap protein in the baculo system. DNA needs to be transfected to SF9 cells for Baculovirus generation. Cap2/5GAr baculo contructs were assigned pvd numbers, which are shown in table 4.

TABLE 2
GAr insertion primer description. This table describes sense and antisense
primer sequences and their respective insertion site in the VP3 protein. The number
represents the location of the insertion in the amino acid chain of Cap2/5 VP3.

| | | Insertion | sense/ | SEQ |
|---|---|---|---|---|
| no# | Sequence | Site | antisense | ID No: |
| Pr243 | GattccacgtggatgGGTGGTGCcGGTGCcGGTGCcGGTggggacagagtcgtc | GAr 236 | sense | 29 |
| Pr244 | Gacgactctgtccccaccggcaccggcaccggcaccacccatccacgtggaatc | GAr 236 | antisense | 30 |
| Pr245 | CaaaagcggctccGGTGGTGCcGGTGCcGGTGCcGGTgtcgacggaagcaac | GAr 267 | sense | 31 |
| Pr246 | Gttgcttccgtcgacaccggcaccggcaccggcaccaccggagccgcttttg | GAr 267 | antisense | 32 |
| Pr247 | GaaccgcgacaacacaGGTGGTGCcGGTGCcGGTGCcGGTgaaaatcccaccgag | GAr 382 | sense | 33 |
| Pr248 | Ctcggtgggattttcaccggcaccggcaccggcaccacctgtgttgtcgcggttc | GAr 382 | antisense | 34 |
| Pr249 | GgcaacaactttgagGGTGGTGCcGGTGCcGGTGCcGGTtttacctacaactttg | GAr 409 | sense | 35 |
| Pr250 | Caaagttgtaggtaaaaccggcaccggcaccggcaccaccctcaaagttgttgcc | GAr 409 | antisense | 36 |
| Pr251 | GtgagcacaaataacGGTGGTGCcGGTGCcGGTGCcGGTactggcggagtccag | GAr 454 | sense | 37 |
| Pr252 | Ctggactccgccagtaccggcaccggcaccggcaccaccgttatttgtgctcac | GAr 454 | antisense | 38 |
| Pr253 | CagttcaacaagaacGGTGGTGCcGGTGCcGGTGCcGGTctggccgggagatac | GAr 467 | sense | 39 |
| Pr254 | Gtatctcccggccagaccggcaccggcaccggcaccaccgttcttgttgaactg | GAr 467 | antisense | 40 |
| Pr255 | CgcgccagtgtcagcGGTGGTGCcGGTGCcGGTGCcGGTgccttcgccacgacc | GAr 502 | sense | 41 |
| Pr256 | Ggtcgtggcgaaggcaccggcaccggcaccggcaccaccgctgacactggcgcg | GAr 502 | antisense | 42 |
| Pr257 | CtatgatcttcaacagcGGTGGTGCcGGTGCcGGTGCcGGTcagccggcgaaccc | GAr 545 | sense | 43 |
| Pr258 | Gggttcgccggctgaccggcaccggcaccggcaccaccgctgttgaagatcatag | GAr 545 | antisense | 44 |
| Pr259 | GgcaccaccgccacgGGTGGTGCcGGTGCcGGTGCcGGTtacctcgagggcaac | GAr 553 | sense | 45 |
| Pr260 | Gttgccctcgaggtaaccggcaccggcaccggcaccacccgtggcggtggtgcc | GAr 553 | antisense | 46 |
| Pr261 | CcggcacgtacaacGGTGGTGCcGGTGCcGGTGCcGGTctccaggaaatcgtg | GAr 596 | sense | 47 |
| Pr262 | Cacgatttcctggagaccggcaccggcaccggcaccaccgttgtacgtgccgg | GAr 596 | antisense | 48 |
| Pr263 | CaccagcttctcgGGTGGTGCcGGTGCcGGTGCcGGTgacgtgcccgtcagc | GAr 663 | sense | 49 |
| Pr264 | Gctgacgggcacgtcaccggcaccggcaccggcaccacccgagaagctggtg | GAr 663 | antisense | 50 |
| Pr265 | CaactacaacgaccccGGTGGTGCcGGTGCcGGTGCcGGTcagtttgtggactttg | GAr 708 | sense | 51 |
| Pr266 | Caaagtccacaaactgaccggcaccggcaccggcaccaccggggtcgttgtagttg | GAr 708 | antisense | 52 |
| Pr280 | CttacccgaccccttGGTGGTGCcGGTGCcGGTGCcGGTtaagacccagctttcttg | GAr 751 | sense | 53 |
| Pr281 | caagaaagctgggtcttaaccggcaccggcaccggcaccaccaaggggtcgggtaag | GAr 751 | antisense | 54 |

TABLE 3
DNA sequencing primers. This table shows the primes sequences used for
DNA sequencing of GAr constructs and their annealing site on the Cap2/5 gene. The
GAr constructs for which these primers can be used is also given in this table.

| | | | SEQ |
|---|---|---|---|
| No# | Primer sequence | Annealing site | ID No: |
| Pr286 | GGACTCCAAGCCTTCCACCTC | gar AAV2/5 sequence primer loc. Cap2/5 | 55 |
| | | gene 480-500 g236 en g267 | |
| Pr287 | Gccaacaacctcacctccac | gar AAV2/5 sequence primer loc. Cap2/5 | 56 |
| | | gene 976-995 g382, 454, 467, 502 | |
| Pr288 | CCAGGGCAGCAACACCTATG | Gar AAV2/5 sequence primer loc. Cap2/5 | 57 |
| | | gene 1551-1570 g553, 596 663 | |
| Pr299 | GCAGGTCACCGTGGAGATG | Gar AAV2/5 sequence primer loc. Cap2/5 | 58 |
| | | gene 2001-2019 g708, 751 | |

TABLE 4
pvd numbers given to baculo expressing Cap2/5 GAr constructs.
This table gives the pvd numbers given to the GAr constructs
with different insertion sites.

| | | Cap2/5 insertion |
|---|---|---|
| Pvd no# | | 5 place |
| pvd121 | Cap2/5 - g236 | 226 |
| pvd122 | Cap2/5 - g267 | 255 |
| pvd123 | Cap2/5 - g382 | 377 |
| pvd124 | Cap2/5 - g454 | 444 |
| pvd125 | Cap2/5 - g467 | 453 |
| pvd126 | Cap2/5 - g502 | 488 |
| pvd127 | Cap2/5 - g663 | 652 |
| pvd128 | Cap2/5 - g708 | 697 |
| Pvd131 | Cap2/5 - g751 | 726 |

The number represents the amino acid insertion site in the VP3 protein.

### Baculovirus Generation Using the FlashBAC™ System

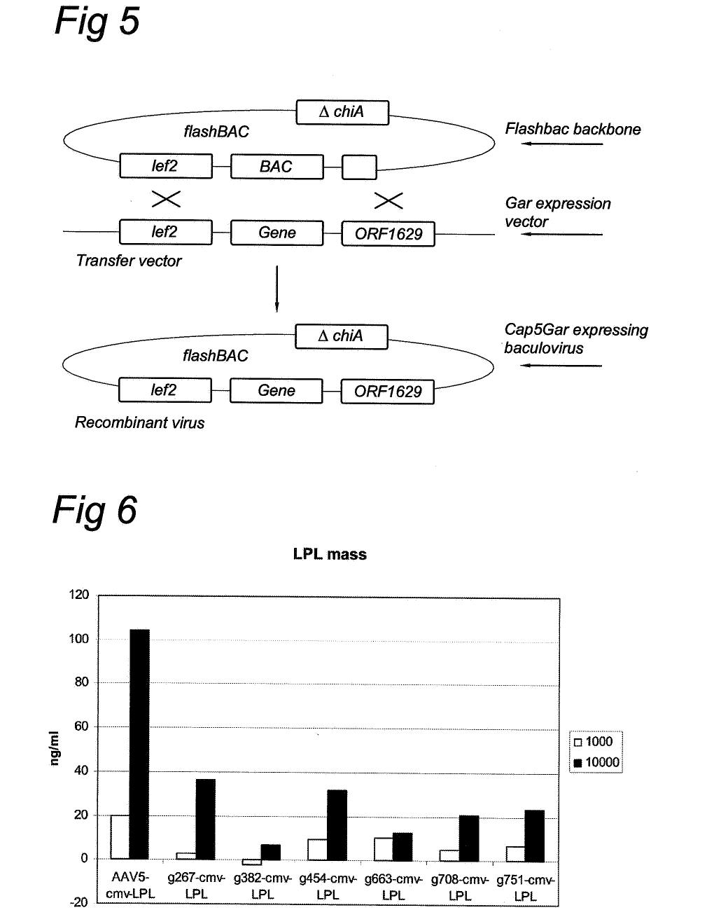

The Cap2/5GAr baculo expression vectors made by the Gateway recombination can now be used to create baculoviruses. These baculoviruses that express Cap2/5GAr proteins were generated using the FlashBAC™ system (NextGen Sciences, Huntingdon, United Kingdom). 100 ng of FlashBAC™ DNA and 500 ng of Cap2/5GAr plasmid (table 4) were transfected into SF9 cells using Cellfectin (Invitrogen). Transfections were performed according to the NextGen Sciences' specifications. 6 hours after the introduction of lipid complexes onto the cells complexes where removed and replaced by sf900II medium (Gibco) supplemented with 10% Fetal bovine serum (Gibco). 5 days post transfection the initial seed stock was harvested by centrifugation (15′ at 1900×g) and the supernatant was stored at 4° C. FIG. 5 shows a graphical representation of the FlashBAC™ recombination reaction. Following homologous recombination of the expression vector into the FlashBAC™ backbone, the initial seed stock should not contain any wild type baculovirus. This is because the expression vector contains an essential gene that is necessary for the replication of baculoviruses. In the FlashBAC™ backbone this essential gene is partially deleted, which prevents replication of wild type baculovirus. Therefore only a baculovirus backbone that has recombined with the Cap2/5GAr expression vector will be able to replicate. The biggest advantage of this vector system is that it does not require a time consuming plaque purification of the initial seed baculo stock.

After harvest of the initial seed stock, one round of amplification for the production of AAV2/5 with a modified capsid gene was performed. To amplify baculovirus to passage 1 (P1), 1 round of amplification was performed. Each round of amplification consists of the following steps: To 50 ml log phase SF+ cells (2.0.10⁶ cells/ml) cultured at 28° C. in SF900 II medium (Gibco) without FBS, 500 ul of initial seed stock (or P1 in a 1:100 ratio) was added to amplify virus. 3 Days post infection virus was harvested using centrifugation (15′ 1900×g, 4° C.). Supernatant is stored at 4° C. and holds the amplified baculovirus. Following each round of amplification the viability of the infected culture was measured on a Nucleocounter (Chemtec). A P1 for each baculo expressing construct was preserved in liquid N₂

To preserve amplified baculovirus for AAV productions, a N₂ freezer stock of P2 baculovirus was created. 2 ml of 100% DMSO (Sigma, cell culture grade) was added to 20 ml of Baculovirus P2. Baculovirus-10% DMSO was then aliquoted in cryovails and snap frozen in liquid N₂. Frozen baculostocks are stored in a N₂ freezer.

### AAV Production of AAV2/5GAr

To produce AAV2/5GAr, 2.0.10⁶ log phase SF+ cells/ml where infected with baculovirus originating from constructs pvd 88 (Rep), Cap2/5GAr and pvd 129 or pvd 43 (hAAT-Apoa1 or CMV-LPL as a transgene). The pvd 88 construct comprises the AAV2 Rep78/52 ORF (modified at the Rep78 initiation codon ATG to ACG) under the control of the PolH insect cell promoter. Both the pvd 129 construct and the pvd 43 construct comprises the a cassette, which is packaged into AAV particles through the ITRs present on the cassette. The pvd 129 cassette comprises the ApoAI gene with its enhancers, a polyA site and two AAV ITRs, whereas the pvd 43 construct comprises the CMV-LPL-WPRE-polyA expression unit between two AAV2 ITRs. Bac.vd 88 (Rep) was added in a 1:20 ratio whereas the transgene and Cap2/5gar baculoviruses were both added in a 1:100 ratio. Infected cells were cultured in SF900II medium (Gibco) without FBS for 3 days at 28° C. AAV2/5GAr viral cultures were lysed by adding 10% of 10× Lysisbuffer (1.5M Nacl, 0.5M Tris-Hcl, 1 mM MgCl₂, 1% Triton x-100, pH=8.5) to the culture and incubating for 1 hour at 28° C. in a shaker incubator. Genomic DNA was digested by adding 4 μl/100 ml Benzonase (Merck) and incubating at 37° C. for 1 hour. Virus was harvested by centrifugation (15′ at 1900×g). Supernatant containing virus was stored at 4° C. Viral titers were determined by Q-PCR against either the hAAT or CMV promoter. Q-PCRs were performed according to standard operating procedures. In short, 5 μl of the AAV comprising sample was added to 45 μl PBS supplemented with 244 μg/ml DNAse (Roche cat. no. 11284932001) and incubated for 20 minutes at 37° C. Subsequently, 75 μl of Proteinase K solution (2.76 mg/ml Proteinase K in Proteinase K buffer) was added and incubated for 60 minutes at 37° C. DNA was then purified from the sample using the magnesil Blue reagents from Promega (Promega, cat. no. A2201, Promega Notes 75 (2000) 7-9). Q-PCR mix was made using the SYBR Green PCR Master Mix (Applied Biosystems, cat. no. 4309155) according to the instruction of the manufacturer (4309155 rev. E).

### AAV2/5GAr Test Production

To determine whether or not AAV2/5GAr could be packaged into viral particles, test productions were run using the baculoviruses that are able to express GAr modified capsid proteins. AAV2/5GArs are produced by combining three baculoviruses. These viruses are able to express Rep (pvd88), a Cap2/5GAr and a baculo that is able to express a transgene (either pvd129 or pvd43 expressing hAAT-apolipoprotein al (hAAT-apoa1) or CMV-lipoprotein lipase (CMV-LPL) respectively). Baculovirus infections are performed in log phase insect cells in a 1:1:5 ratio of Cap2/5GAr, transgene and Rep. 3 days post infection the AAV's are harvested by lysing the cells. Viral titers are determined in the crude lysate using a Q-PCR assay as has been described above.

Viral titers of AAV2/5GAr varied between 3.5.10⁸-2.2.10¹⁰ genome copies (gc)/ml for virus produced with Apoa1 as a transgene and 5.10⁸-1.9.10¹⁰ gc/ml for LPL^{S447X} as a transgene. These data suggest that AAV2/5 modified with GAr insertions at different locations in the VP3 protein are able to package. The location of the insertion does not seem to affect the viral titers. To further access the infectivity of these modified AAV2/5GArs in vivo and in vitro experiments are carried out. Viral titers of the test productions are summarized in table 5.

TABLE 5
Viral titers in gc/ml of test AAV2/5GAr
test productions determined by Q-PCR.

| Capsid | apoa1 (pvd129) | lpl (pvd43) |
|---|---|---|
| Cap2/5 g236 | 3.35.10⁹ | 1.25.10⁹ |
| Cap2/5 g267 | 1.11.10⁹ | 4.75.10⁸ |
| Cap2/5 g382 | 5.37.10⁸ | 1.34.10⁹ |
| Cap2/5 g454 | 2.95.10⁹ | 1.85.10¹⁰ |
| Cap2/5 g467 | 5.14.10⁸ | 5.34.10⁸ |
| Cap2/5 g502 | 1.09.10⁹ | 4.79.10⁸ |
| Cap2/5 g663 | 2.23.10¹⁰ | 1.01.10⁹ |
| Cap2/5 g708 | 3.09.10⁹ | 6.30.10⁸ |
| Cap2/5 g751 | N/D | 5.20.10⁸ |
| Cap2/5 | 3.78.10⁸ | 1.90.10⁹ |

N/D: not determined

### Conclusion

The above data suggest that AAV2/5 modified with GAr insertions at different locations in the VP3 encoding nucleotide sequence are able to package. The location of the insertions did not seem to affect the viral titers. This was mostly due to the large spread of viral titers between the two transgenes used for the same insertion site. To access the infectivity and functionality of modified AAV2/5GArs, in vivo and in vitro experiments were carried out.

### Example 2

### AAV Production and Purification

To produce AAV2/5GAr, 2.0.10⁶ log phase SF+ cells/ml were infected with baculovirus originating from constructs pVD88 (Rep), Cap2/5GAr and pVD129 or pVD43 (hAAT-Apoa1 or CMV-LPL as a transgene). Bac.VD88 (Rep) was added in a 1:20 ratio whereas the transgene and Cap2/5GAr baculoviruses were both added in a 1:100 ratio. Infected cells were cultured in SF900II medium (Gibco) without FBS for 3 days at 28° C. AAV2/5GAr viral cultures were lysed by adding 10% of 10× Lysisbuffer (1.5M Nacl, 0.5M Tris-Hcl, 1 mM MgCl₂, 1% Triton x-100, pH=8.5) to the culture and incubating for 1 hour at 28° C. in a shaker incubator. Genomic DNA was digested by adding 4 μl/100 ml Benzonase (Merck) and incubating at 37° C. for 1 hour. Virus was harvested by centrifugation (15′ at 1900×g). Supernatant containing virus was stored at 4° C. Prior to loading crude lysate onto the affinity column it is filtered on a 0.45 μm Millipak filter (Millipore). Viral titers were determined by Q-PCR against either the hAAT or CMV promoter. Q-PCR's were performed as has been described above, using AMT primers 300-301 and 59-60 specific for the hAAT and CMV promoter respectively.

AAV2/5GAr was purified by affinity chromatography on an AKTA explorer system (GE healthcare). AAV2/5GAr was eluted from 1 or 5 ml AVB sepharose (GE healthcare) columns using PBS pH=3.0 and pH=2.0 (Gibco) which was immediately buffered in 1M Tris-HCl pH=8.5 (Sigma) after elution. To concentrate virus to a final concentration of 1.10¹² gc/ml virus was first diafiltered to a 200 mM PO4 pH=7.5 0.01% Pluronic F68 buffer (prevents aggregation as well as binding to plastic) on a 400 kd 615 cm² hollow fiber (JM separations). Next, virus was further concentrated in centricon tubes with a cut off of 100000 MCW (Millipore). To change the buffer to a suitable buffer for in vivo experiments an overnight dialysis was performed in 5 ml dialysis membranes with a cut off of 100000 MCW (Spectrum labs). Dialysis was performed overnight to 1 L of PBS-5% sucrose (Gibco) Dialysis buffer was changed twice. To sterilize the sample was filtered on a 0.22 μm Millex GP filter (Millipore). Viral titers in final concentrate were determined as has been described above, using AMT primers 300-301 and 59-60 specific for the hAAT and CMV promoter respectively. For experiments using AAV2/5GAr-cmv-lpl, virus was eluted in PBS pH=3.0 and 2.0, but no further buffer exchange or concentration was performed.

### In Vitro Infectivity

For initial in vitro experiments performed with AAV2/5GAr, six out of nine available baculo constructs were selected for AAV2/5GAr production (Cap2/5-267, Cap2/5-382, Cap2/5-454, Cap2/5-663, Cap2/5-708, Cap2/5-751). These six Cap2/5GAr constructs gave the highest average viral titer during multiple test productions. AAV2/5GArs were made with ApoA-1 and LPL as transgenes and were subsequently purified by affinity chromatography. Purified AAV2/5GAr stocks were used for in vitro infectivity experiments. Infectivity was initially assessed by LPL mass ELISA. Following the ELISA one AAV2/5GAr was selected for in vivo experiments with ApoA-1 as a transgene (AAV2/5GAr267, this AAV2/5GAr gave the highest infectivity in the LPL mass ELISA). ApoA-1 infectivity in Hela cells was determined via a new method. In this assay the total amount of woodchuck post-transcriptional regulatory element (WPRE) single strand DNA (ssDNA), which is only found on our ApoA-1 vector, is measured in the nucleus by Q-PCR. Infectivity is a measure of the amount of WPRE DNA found in the nucleus compared to the amount in the cytoplasm.

### LPL Mass Infectivity Assay

LPL mass infectivity assay was performed according to the instructions of the LPL mass activity kit from DS Pharma Biomedical (Osaka, Japan, #2009.6 0611). With the distinction that instead of HEK 293 cells, 84-31 cells (Fisher et al. (1996) J. Virol 70:520-532) were used. These cells are derived from the HEK 293 cell line and contain the E1 and E4-regions from Ad-5. This eliminates the need to co-transfect cells with wt-ad, which gives a better indication of infectivity of AAV2/SGAr. 5.10⁵ 84-31 cells were infected with AAV2/SGAr at MOIs varying between 1000 and 10000. 24 hours post transfection supernatant was harvested and used to determine LPL mass with a Markit-M LPL kits (DS Pharma Biomedical (Osaka, Japan, #2009.6 0611)). Absorbance was measured at 492 nm in a Softmax Pro (Molecular devices).

### ApoA-1 WPRE Infectivity

5.10⁵ Hela cells were infected at MOIs of 10⁴ and 10⁵ with purified AAV2/5 or AAV2/SGAr stocks that contained ApoA-1-WPRE (pvd129) as a transgene. Cells were co-transfected with 5.10⁵ ifu of wt-ad. 72 hours post transfection the nuclei and cytoplasm of infected cells were harvested using the Nuclei Isolation Kit: Nuclei EZ Prep (Sigma). Isolations were performed according to the manufacturer's specifications with the exception that all volumes used are divided by two. This is because a smaller amount of cells is used for the isolation. Next, DNA is isolated from the nuclei and cytoplasm by using the Easy DNA kit protocol nr. 3 (Qiagen). With the exception that DNA was precipitated overnight at −20° C. To prevent a high background during the Q-PCR caused by RNA the DNA dissolved in 10 mM Tris-HCl supplemented with RNAse.

Before Q-PCR the DNA concentration was measured on a Nanodrop. 16.6 ng of Cytoplasm or Nuclei DNA was added to each reaction. Q-PCR was run on a 7000 Abi prism system program:

Primers were designed that bind specifically to the WPRE enhancer, which element can only be found in the DNA packaged by the virus. Primers are described in table 6. Reaction mix: 10 pmol Forward and reverse primers, 16.6 ng of nuclear or cytoplasm DNA. Reaction was brought to its final concentration by using 2× Sybr Green master mix (Applied Biosystems). Q-PCR program: 10′ at 95° C., followed by 40 cycles of 15″ at 95° C. and l′ at 60° C.

TABLE 6
WPRE Q-pcr with primers.

| | | Forward/ | SEQ |
|---|---|---|---|
| Pr no# | Primer sequence | reverse | ID NO: |
| pr315 | GCTGCTTTAATGCCTTTGTATC | wpre forward | 59 |
| pr316 | AATGAAAGCCATACGGGAAG | wpre reverse | 60 |

### Results

### AAV Production and Purification

In vitro experiments with AAV2/5GAr are performed with purified rAAV stocks. To produce AAV2/5GAr three baculoviruses were combined. These viruses are able to express Rep (Bac.VD88), a Cap2/5GAr (Bac.VD121-131) and a baculo that is able to express a transgene (either Bac.VD129 or Bac.pVD43 expressing hAAT-apoa1-WPRE or CMV-LPL respectively). Baculovirus infections are performed in log phase insect cells in a 1:1:5 ratio of Cap2/5, transgene and Rep. 3 days post infection the AAV's are harvested by lysing the cells. Viral titers are determined in the crude lysate by Q-PCR assay as has been described above. For AAV2/5-cmv-lpl 440 ml of crude lysate was produced. Stocks for this assay did not need any further concentration and eluate directly from the column was used for the experiments. For the AAV2/5-hAAT-Apoa1 productions 4400 ml of crude lysate was produced. These stocks were concentrated to a final concentration of ca. 1.10¹² gc/ml. Recovery's of eluates per construct are summarized in table 7 and 8.

TABLE 7
Overview 440 ml AAV2/5GAr cmv lpl productions.

| | Crude | ph = 3.0 | ph = 2.0 | |
|---|---|---|---|---|
| | lysate | eluaat | eluaat | % |
| | gc/ml | gc/ml | gc/ml | recovery |
| G267-cmv-lpl | 4.03.10⁹ | 3.63.10¹⁰ | 4.69.10⁹ | 45.6 |
| G382-cmv-lpl | 6.05.10⁹ | 5.06.10¹⁰ | 2.17.10¹⁰ | 59.7 |
| g454-cmv-lpl | 9.27.10⁹ | 7.23.10⁹ | 8.40.10⁹ | 84.3 |
| G663-cmv-lpl | 2.02.10⁹ | 6.22.10⁷ | 7.58.10⁹ | 18.9 |
| g708-cmv-lpl | 1.02.10¹⁰ | 1.55.10¹⁰ | 4.95.10¹⁰ | 31.9 |
| g751-cmv-lpl | 4.73.10⁹ | 9.49.10⁹ | <1.10⁷ | 10.0 |
| AAV2/5-cmv-lpl | 1.11.10¹⁰ | 9.64.10¹⁰ | 8.23.10¹⁰ | 80.5 |

% recovery is the recovery in the elution of the AAVs from the Llama column.

TABLE 8
Overview 4400 ml AAV2/5GAr-hAAT-Apoa-1 productions.

| | crude lysate | final stock | | % |
|---|---|---|---|---|
| | gc/ml | gc/ml | capsid/ml | recovery |
| AAV2/5-hAAT- | 4.40.10⁹ | 1.10.10¹² | 8.43.10¹³ | 25% |
| ApoA-1 | | | | |
| AAV2/5g267- | 8.38.10⁹ | 1.10.10¹² | 2.74.10¹³ | 15% |
| hAAT-ApoA-1 | | | | |

% recovery is the recovery for the total purification process.

### LPL Mass Infectivity Assay

To determine whether or not the insertion of GAr at different sites in the Cap2/5 gene affects the infectivity of virus particles we assayed LPL mass activity of AAV2/5GAr that had insertions at several different sites.

To this end we produced and purified six AAV2/5GArs and one AAV2/5 control with LPL as their transgene. Virus was only purified by affinity chromatography and as the column eluate was directly used for the infectivity assay.

For the infectivity assay we used the 84-31 cell line. This line is derived from the HEK 293 cell line and is stably transfected with epitopes originating from wild type Adenovirus (ad-1 and ad-5). Co-transfection with wild-type adenovirus (wt-AD) is not required when using this cell line and therefore prevents unwanted background. AAV will enter the cell in the presence of wt-AD regardless of GAr. And it is the effect of GAr on the ability of the AAV particle to enter cell that we want to test. Use of this cell line allows for distinction of infectivity between different GAr insertion locations.

84-31 cells were infected with purified six AAV2/5GAr stocks at MOIs between 1000-10000. 24 hours post infection supernatant was harvested and assayed for total LPL mass by an ELISA kit.

Results from this assay are summarized in FIG. 6. As can be seen AAV2/5 has an infectivity about 3 fold higher than the best GAr construct. This could be caused by the alteration of the binding domains of GAr to the or the folding of GAr.

### ApoA-1 WPRE Infectivity

The ApoA-1 ELISA used to determine ApoA-1 activity in vivo, could not be used for cells that were infected in vitro. As an alternative to this assay we developed a method that detects the amount of AAV2/5GAr transgene DNA in the nucleus. DNA from complete nuclei was isolated and Q-PCR was carried out for the presence of the WPRE enhancer. This enhancer is only found on the transgene of our ApopA-1 construct. The amount of WPRE DNA found in the nucleus is an indication of the amount of infective cells.

Cells were infected at MOIs of 1.10⁴ and 1.10⁵ of AAV2/5GAr267 or AAV2/5 without minimal GAr region together with wild-type-adenovirus. 24 hours post transfection nuclei were isolated from infected cells. Cytoplasma from lysed cells was also stored for this assay. DNA from nuclei and cytoplasma was isolated and next the number of WPRE copies present in these samples was assayed using Q-PCR against WPRE. WPRE is only present on our ApoA-1 construct and can therefore function as marker for infectivity of the modified AAV2/5 particle. Difference between the amount of WPRE found in the cytoplasm and nucleus is a measure of infectivity for the AAV2/5GAr particle as a whole. This assay can however not determine whether or not the ApoA-1 transgene was expressed by the cell.

Infectivity is about 10 times lower in cells infected with AAV2/5GAr when compared to cells infected with unmodified capsids. These results are similar to the ones obtained with the LPL mass infectivity assay.

### Conclusion

This example shows that the AAV2/5GArs that are produced by the Cap2/5GAr baculo constructs are infective in HEK 84-31 and HELA cells. The AAV2/5GArs can be used to access functionality of GAr both in vivo and in vitro.

### Example 3

### Infectivity of the rAAV-GAr Vector In Vivo

First, the in vivo infection efficiency of cells by the rAAV-GAr vectors was tested. The rAAV2/5-GAr vectors containing eGFP (enhanced Green Fluorescent Protein) expression cassette were injected intravenously into C57/b16 or BALB/c mice and the transgene expression was measured. Approximately 6×10¹² genomic copies of rAAV-GAr are injected per kg mouse. The eGFP expression was analyzed by microscopic analysis and/or immunohistochemistry of the major organs focussing on the liver and spleen.

Tissue processing was carried out as follows. For fluorescent microscopy, the tissues or fractions of tissue were fixed by immersion with 4% formaldehyde/7% picric acid/10% sucrose in PBS, rinsed quickly with PBS, frozen in liquid nitrogen and stored in −80° C. until cryosectioning. For immunohistochemistry fractions of liver, spleen and thymus were frozen in liquid nitrogen immediately after preparation and stored in −80° C. until cryosectioning.

Fluorescent microscopy was carried out as follows. Sections were cut in a cryostat (Leica) at 7 μm. After washing with PBS the sections were mounted with hardening Vectashield containing DAPI.

In the first experiment, GARr constructs 267, 382, 454, 663, or 708 were used (see Tables 1 and 9). The mice were sacrificed at day 14 and liver, spleen, lymph nodes, kidney, intestine (proximal part), testis muscle, heart, lung, thymus and blood were collected.

TABLE 9
Experimental set up: 6 groups, 5 mice by group

| | |
|---|---|
| Mouse 1-5 | AAV5-hAAT-eGFP (wt) |
| Mouse 6-10 | G267 AAV5-hAAT-eGFP |
| Mouse 11-15 | G382 AAV5-hAAT-eGFP |
| Mouse 16-20 | G454 AAV5-hAAT-eGFP |
| Mouse 21-25 | G663 AAV5-hAAT-eGFP |
| Mouse 26-30 | G708 AAV5-hAAT-eGFP |

In the second experiment, GARr constructs 267, 382, 454, 663, or 708 were used (see Tables 1 and 10). The mice were sacrificed at either day 14 or day 21 and the liver, spleen, thymus, lymphoid nodes and blood were collected.

TABLE 10
Experimental set up: 4 groups, 10 mice by group, 5 mice
sacrificed 14 days after injection, 5 mice at 21 days.

| | Sacrificed | Sacrificed |
|---|---|---|
| Group | at 14 days | at 21 days |
| 1. AAV5-hAAT-eGFP (wt) | Mouse 1-5 | Mouse 21-25 |
| 2. G267 AAV5-hAAT-eGFP | Mouse 6-10 | Mouse 26-30 |
| 3. G382 AAV5-hAAT-eGFP | Mouse 11-15 | Mouse 31-35 |
| 4. PBS | Mouse 16-20 | Mouse 36-40 |

Fluorescent microscopy of liver sections showed that the GFP expression levels could be detected for all of the GAr constructs tested (data not shown). Noticeable is that the GFP expression was higher in the pericentral areas than in the periportal areas of the liver. This was observed in both the wt as well as the GAr constructs.

Based on the results of the first experiment, two GAr constructs were selected for further testing: G267 AAV5-hAAT-eGFP and G382 AAV5-hAAT-eGFP. Fluorescent microscopy of liver sections from the second experiment showed results similar to those obtained in the first experiment I. The mice that were sacrificed 21 days after injection of the wt construct showed a stronger intensity of GFP expression than the mice that were sacrificed after 14 days which reflects an accumulation of the GFP protein in the cell.

### Example 4

### Ex Vivo Measurement of the GAr Function by In Vitro Neutralization Antibody Assays

In order to test the biological efficacy of the Gar constructs, in vitro neutralisation antibody assays were carried out using blood samples collected in the experiments described in Example 3.

Plasma was collected by spinning the blood samples for 5 min at 7000 RPM and then analysed by neutralising antibody assays.

In the neutralizing antibodies assay, HEK293 cells (CRL-1573, ATCC) passage x+32 were seeded in a 96 wells plate (Ultraweb, Corning) at a density of 2e5 cells/well in 100 μl DMEM (Gibco) with 10% FBS and antibiotics (P/S) and incubated overnight at 37° C.

2.10⁹ gc's AAV5.cmv.GFP (vd.92.88.138 lotnumber: A0212-006) were incubated with serum sample (pre-inactivated by 1 hour heating at 56° C.) and with wild type-adenovirus (A0168-162) at a dilution of 3:2000 in a total volume of 200 μl of DMEM. The mix was kept for 1 hour at 4° C. before being added.

The medium of the HEK293 cells was removed by aspiration and the mix of serum, AAV5.cmv.GFP and adenovirus was added for 20 hr at 37° C. The final dilution of the test serum was 1:100 and 1:1.000. The cells were collected after trypsinisation and washed in PBS with 1% (w/v) BSA. Cellular GFP expression was analyzed by fluorescence-activated cell sorting (FACScalibur, Becton Dickinson). The analysis was performed with the Cellquest software. The percentage of inhibition was calculated related to GFP expression measured in AAV5.cmv.GFP infected HEK293 cells (no inhibition, 100% expression).

In the first experiment, neutralizing antibodies were generated after AAV5 wild type injection was measured in mouse plasma at week 2.

Target cells were infected with the AAV5 wild type in presence of mouse plasma. The inhibitory effect on cell infection of the Neutralizing antibodies present in the plasma was monitored.

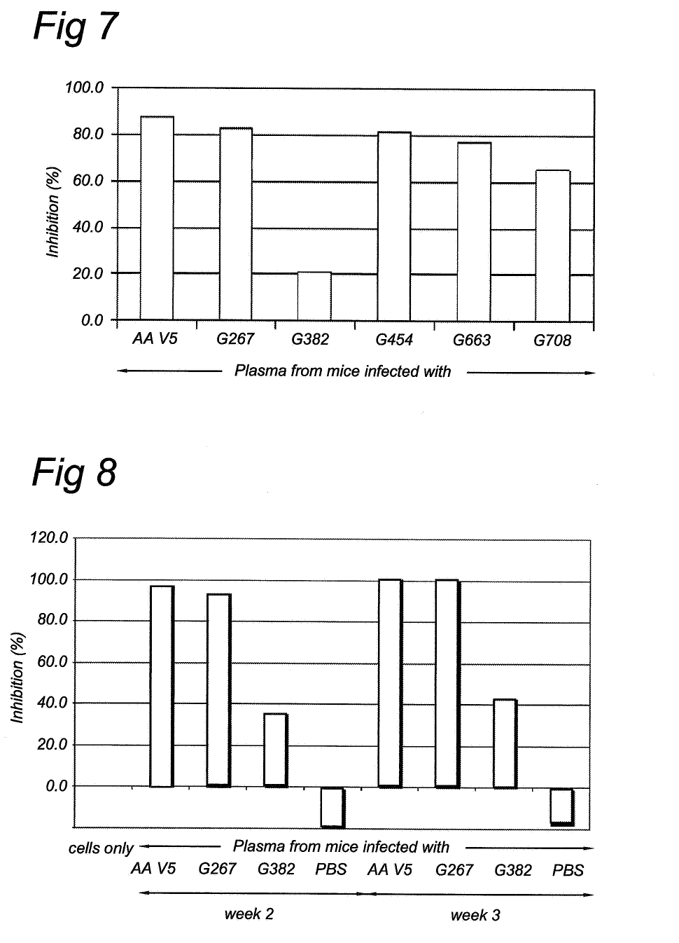

The infection of target cells by the AAV5 wt is significantly less inhibited in presence of G382 plasma (20% of inhibition), than in presence of AAV5 wt plasma (90% of inhibition) and the other GAr plasma (65 to 80%)—see FIG. 7.

This result suggests that neutralizing antibodies raised against the G382 capsid do not prevent in vitro the AAV5 wt to infect cells.

In the second experiment, the inhibitory effects of neutralizing antibodies generated after injection with AAV5 wt and the 2 GAr constructs, G382 and G267 were measured in mouse plasma at week 2 and 3.

The infection of target cells by AAV5 wt is again significantly less inhibited in presence of G382 plasma than in presence of GAr 267 and the AAV5 wt plasma. The decrease in inhibition is stable over time (2 and 3 weeks)—see FIG. 8.

This result confirmed that neutralizing antibodies raised against the G382 capsid do not prevent the AAV5 wt to infect cells in vitro.

To determine the cross effect of the Neutralizing antibodies generated against AAV5 wt, G382 and G267 capsid, Neutralizing antibodies generated after injection were measured in mouse plasma at weeks 2 and 3. Target cells were infected with G382 in presence of mouse plasma. The inhibitory effect of the Neutralizing antibodies on cellular infection was measured.

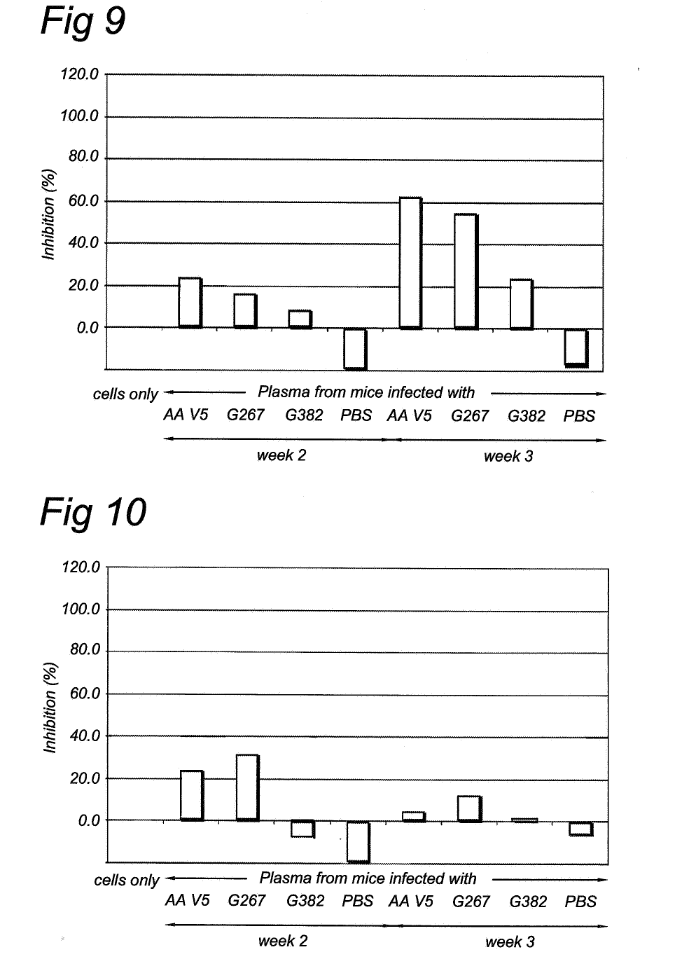

The infection of target cells by G382 was not significantly inhibited at 2 weeks by AAV5 wt, G267 and G382 plasma (20% to 10% of inhibition), but the inhibition seems to become more effective over time (60 to 30% of inhibition at 3 weeks)—see FIG. 9.

This result suggests that neutralizing antibodies raised against the G382 capsid have little effect on cell infection by G382 itself but also that neutralizing antibodies raised against the AAV5 wt and G267 are not very effective in preventing infection by G382. At 2 weeks, neutralizing antibodies raised against AAV5 wt, G267 and G382 capsids do not prevent the GAr construct G382 to infect cells in vitro.

Target cells were infected with the G267 in presence of mouse plasma. The inhibitory effects of the neutralizing antibodies on cellular infection were measured. The infection of target cells by G267 in presence of AAV5 wt and G267 plasma is only slightly inhibited (20% to 30% of inhibition) at 2 weeks, and seems to become even less inhibited over time (5 to 10% of inhibition)—see FIG. 10. No inhibition of cellular infection by G267 was noticeable at 2 and 3 weeks in presence of G382 plasma. This result suggests that neutralizing antibodies raised against the G382 capsid do not prevent the G267 to infect cells in vitro.

The data obtained demonstrate that neutralizing antibodies raised against the GAr382 capsid does not prevent in vitro AAV5 wt from infect cells nor does it prevent the GAr267 from being infective.

As a mirror effect, neutralizing antibodies raised against AAV5 wt, G267 and G382 capsids do not prevent the GAr construct G382 from infecting cells in vitro.

### Example 5

### Ex Vivo Measurement of the GAr Function

The functionality of the GAr insertion is first investigated ex vivo in a cytotoxic T-cell (CTL) cytotoxicity assay. Hereto, CTLs specific for the AAV2/5 capsid are generated by induction of the immune responses to AAV2/5 in mice, according to the different protocols of immunisation described below. Upon immunization with AAV, the CTLs are prepared from the spleens, liver and blood of the mice injected An assay is performed to determine whether the CTLs can kill cells transduced with a normal AAV2/5 (wild type), and cannot kill cells transduced with AAV2/5GAr. This cytotoxicity assay can be performed in several different ways, but all methods look at the CTL function on target cells that present epitopes (or not) that are recognized by the CTLs and that activate the CTLs to kill those cells. This assay can be performed in vitro. Briefly, a target murine cell line will be transduced by AAV2/5 or AAV2/5GAr and subsequently the CTLs generated in vivo will be added. The AAV2/5GAr transduced cells show reduced recognition by CTLs and less killing as compared to the control AAV2/5 transduced cells.

### In Vivo Measurement of the GAr Function

The functionality of the GAr insertion is investigated in vivo by first immunisation of C57/b16 mice (1), followed by transduction by the rAAV2/5-GAr construct containing an expression cassette with a reporter gene (2). Subsequently the reporter gene is measured in time. The level of expression of the reporter gene is higher in the rAAV2/5-GAr vector injected animals than in the rAAV2/5 control (without GAr) injected animals, because of the greater loss of expression due to the immune responses in control animals as compared to rAAV2/5-GAr treated animals.

Immunisation is done by several different immunisation protocols. In one of the protocols the mice are immunised with Mannan (mannose based) coated rAAV2/5 to direct the rAAV2/5 specifically towards the dendritic cells and improve the presentation. In another protocol the mice are immunized by intramuscular injection of a adenovirus comprising an expression cassette of the AAV2/5 capsid proteins (the so-called prime), followed 14 days later by an intravenous injection of an AAV2/5 vector (the so-called booster).

### Redirecting the Adenovirus Used for Immunization to Dendritic Cells; Mannan Coating

Distribution of AAV2/5-GFP, mannan-conjugated AAV2/5-GFP, Ad5-GFP and mannan-conjugated Ad5-GFP to the liver and to dendritic cells in the spleen in Balb/c mice is compared after intraperitoneal administration (table 11). The mannan modification is required to demonstrate whether dendritic cells can be targeted with this modified vector, thus enabling the study of vector-specific T-cell responses. This forms the basis for an immunomodulatory approach to AAV2/5-based gene therapy in the liver and provides a method to induce AAV-directed immune responses to this serotype. The read-out is based on localisation of GFP expression in liver, spleen, and surrounding tissues, and co-localisation of the GFP with the dendritic cell marker CD11c. CD4+ and CD8+ T cell responses are monitored by using specific markers.

TABLE 11
redirecting adenovirus used for immunization to dendritic cells

| | | | | | Administration | | |
|---|---|---|---|---|---|---|---|
| Group | Strain | Vector | Sex | Amount | route | Dose | |
| 1 | Balb/c | AAV2/5-GFP | ♀ | 5 | i.p. | 1^{E}13 | gc/ml |
| 2 | Balb/c | Man. AAV2/5-GFP | ♀ | 5 | i.p | 1^{E}13 | gc/ml |
| 3 | Balb/c | Ad5.GFP | ♀ | 5 | i.p | 1e8 | pfu |
| 4 | Balb/c | Man. Ad5.GFP | ♀ | 5 | i.p | 1e8 | pfu |

| | | | | | | |
|---|---|---|---|---|---|---|
| 5 | Balb/c | PBS | ♀ | 5 | i.p | n/a |

### Immunization and Development of Memory CTLs

Mice are immunized, following one of the protocols precedently described with or without the use of soluble CD83 injections to inhibit formation of neutralizing antibodies against the AAV 5 capsid. The immunization is repeated every 2 weeks for up to 5 times, and subsequently memory CTL are allowed to develop for at least 3 months to 6 months. Subsequently, AAV2/5-GAr or the control AAV2/5 is intravenously or intraperitoneally injected, and analysis is carried out to monitor whether the memory CTL's are activated by the control AAV2/5, and not by the AAV2/5-GAr, leading to loss of reporter gene expression in the AAV2/5 control injected animals, but not in the AAV2/5-GAr injected animals. Activation of the memory CTL's is monitored by several ways, including the CTL assay mentioned above.

1. A nucleic acid construct comprising a nucleotide sequence encoding parvoviral VP1, VP2, and VP3 capsid proteins, wherein the nucleotide sequence comprises at least one in-frame insertion of a sequence coding for an immune evasion repeat, that is a amino acid sequence that comprises 1, 2 or 3 units of a formula (Glyₘ-Xaa₁-Glyₙ-Xaa₂-Glyₚ-Xaa₃-Gly_{q}), wherein
m and q are independently 0, 1 or 2,
n and p are independently 1, 2 or 3,
each of m, q, n and p are chosen such that the immune evasion repeat consists of at least 8 amino acids, and
each of Xaa₁, Xaa₂, and Xaa₃ are independently Ala, Val or another small hydrophobic amino acid residue.

2. A nucleic acid construct according to claim 1, wherein
one of m, n or p is 2,
the other two of m, n or p are 1,
q is 1, and
Xaa₁, Xaa₂ and Xaa₃ are Ala.

3. A nucleic acid construct according to claim 1, wherein at least one sequence coding for the immune evasion repeat is present in a VP3 capsid protein-coding part of the nucleotide sequence.

4. A nucleic acid construct according to claim 1, wherein the sequence encoding an immune evasion repeat is positioned such that the encoded immune evasion repeat is present in at least one position in the VP3 capsid protein that encodes amino acids that are immediately N-terminal to AAV2/5 hybrid VP3 capsid protein (SEQ ID NO:61) at position 226, 255, 377, 444, 453, 488, 652, 697 or 726 of SEQ ID NO:61.

5. A nucleic acid construct according to claim 1, wherein the nucleotide sequence is operably linked to expression control sequences for expression of said nucleotide sequence in a mammalian or insect cell.

6. A mammalian or insect cell comprising a nucleic acid construct according to claim 5.

7. A cell according to claim 6, further comprising:
(a) a second nucleotide sequence comprising at least one parvoviral inverted terminal repeat (ITR) nucleotide sequence; and
(b) a third nucleotide sequence comprising a Rep52 or a Rep40 coding sequence operably linked to expression control sequences for expression in the cell; and,
(c) a fourth nucleotide sequence comprising a Rep78 or a Rep68 coding sequence operably linked to expression control sequences for expression in the cell.

8. A cell according to claim 7, wherein the second nucleotide sequence further comprises at least one nucleotide subsequence encoding a gene product of interest for expression in a mammalian cell, which subsequence is incorporated into a genome of a parvoviral virion produced in the cell.

9. A parvoviral virion comprising a capsid protein that comprises at least one immune evasion repeat that is an amino acid sequence that comprises 1, 2 or 3 units of a formula (Glyₘ-Xaa₁-Glyₙ-Xaa₂-Glyₚ-Xaa₃-Gly_{q}), wherein
m and q are independently 0, 1 or 2,
n and p are independently 1, 2 or 3,
each of m, q, n and p are chosen such that the repeat consists of at least 8 amino acid residues, and
each of Xaa₁, Xaa₂, and Xaa₃ are independently Ala, Val or another small hydrophobic amino acid residue.

10. A parvoviral virion according to claim 9, wherein at least one immune evasion repeat is present in a VP3 capsid protein.

11. A parvoviral virion according to claim 10, wherein the sequence encoding an immune evasion repeat is positioned such that the encoded immune evasion repeat is present in at least one position in the VP3 capsid protein that is immediately N-terminal to an amino acid in the VP3 capsid protein that corresponds to amino acid position 226, 255, 377, 444, 453, 488, 652, 697 or 726 as defined with reference to the AAV2/5 hybrid capsid protein as set out in SEQ ID NO: 61.

12.-14. (canceled)

15. A pharmaceutical composition comprising a parvoviral virion according to claim 9 and a pharmaceutically acceptable carrier.

16. A method to reduce an immune response against a gene therapy vector in a subject, comprising administering an effective amount of a parvoviral virion according to claim 9 to a subject in need thereof, thereby reducing said immune response.

17. A method for producing a parvoviral virion, comprising the steps of:
(a) culturing the cell according to claim 6 under conditions such that a parvoviral virion is produced; and,
(b) recovering of the parvoviral virion.

18. The nucleic acid construct according to claim 2 wherein m is 2, n is 1 and p is 1.

19. The method according to claim 16 wherein the subject has detectable T cell immunity against the parvoviral virion prior to said administering.

20. The method according to claim 16 wherein the parvoviral virion is administered to the subject at least twice.
